(19) United States
(12) Patent Application Publication
(10) Pub. No.: US 2007/0092576 A1
(43) Pub. Date: Apr. 26, 2007

(54) Compositions containing opioid antagonists

(75) Inventor: John D. Buehler, Ambler, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Adolor Corporation, Exton, PA (US)

(21) Appl. No.: 11/582,681

(22) Filed: Oct. 18, 2006

Related U.S. Application Data
(60) Provisional application No. 60/728,557, filed on Oct. 20, 2005.

(51) Int. Cl. A61K 31/485; A61K 31/454; A61K 31/452

(52) U.S. Cl. 424/489; 514/317; 514/326; 514/282

(57) ABSTRACT

Compositions containing opioid antagonists are disclosed, particularly alvimopan and its active metabolite in solid dosage forms, where the drug is uniformly distributed, achieves the desired bioavailability, and is stable. Methods of preparing and using the compositions containing opioid antagonists are also disclosed. The results are achieved by a combination of processing techniques and component selection.

### CROSS REFERENCE TO RELATED APPLICATIONS

This application claims the benefit of U.S. application No. 60/728,557 filed Oct. 20, 2005, the entire disclosures of which are incorporated herein by reference.

### FIELD OF THE INVENTION

The present invention relates to compositions containing opioid antagonists. More particularly, the present invention relates to compositions containing opioid antagonists, especially solid dosage forms thereof, and methods of preparing and using them.

### BACKGROUND OF THE INVENTION

[[2(S)-[[4(R)-(3-hydroxyphenyl)-3(R),4-dimethyl-piperidinyl]methyl]-1-oxo-3-phenylpropyl]amino]acetic acid dihydrate (USAN name alvimopan) and its active metabolite are peripherally-acting μ opioid antagonists that may be used in the treatment of postoperative ileus, postpartum ileus, pruritis, constipation, opioid bowel dysfunction, urinary retention, biliary spasm, opioid bowel dysfunction, colic, postoperative nausea, and/or postoperative vomiting as well as other indications. Alvimopan is currently available in solid dosage form. Alvimopan and its active metabolite are 3,4-disubstituted-4-aryl piperidines that are zwitterions. They have extremely low solubility in water and many common pharmaceutically acceptable solvents. Alvimopan is more soluble at an acid pH than a basic pH. Thus, the bioavailability of orally-administered alvimopan may be altered if the patient takes the drug with food or if the patient is receiving therapy to control gastric pH. This low and variable solubility raises concerns both in the manufacture of dosage forms and in the use in vivo to elicit the desired therapeutic effect.

Alvimopan is very potent and, therefore, a patient only requires a very low dose of alvimopan to achieve its therapeutic effect. This very lose dose creates a challenge when the alvimopan is formulated with excipients to ensure the proper level of the drug and its uniformity or homogeneity in any dosage form, especially solid dosage forms. For example, if alvimopan has an average particle size of 0.25 mm and a 0.5 mg dose is required in the solid dosage form, only 40 particles of alvimopan would be required to provide the target dose. Mixing 40 particles of alvimopan with excipients of various sizes and shapes leads to difficulties in achieving the proper dose and uniformity.

Alvimopan and related compounds have not only low water solubility, but are also hydrophobic. With drugs that are hydrophobic and have low water solubility, formulators often include one or more wetting agents and/or surfactants. Unfortunately, the wetting agents and/or surfactants can affect the drug's stability and may not be suitable for consumption.

Another problem that is somewhat unique to alvimopan and related compounds is the stability of the hydration of the molecule. Alvimopan is a 3,4-disubstituted-4-aryl piperidine in dihydrate form. To maintain its effectiveness in vivo, it is desirable to maintain the dihydrate form of the drug. Studies have shown that alvimopan must be maintained under controlled temperature and humidity conditions to avoid changes in the polymorphic structure.

Micronization is a high-energy, dry-milling process that reduces the particle size of drug powders to ultrafine size, typically in the range of one to ten microns. Micronization of a drug to reduce its particle size is known to improve the bioavailability of certain drugs. However, due to the electrostatic charge that is generated during micronization, the drug tends to agglomerate to form larger effective particle sizes thereby reducing dissolution rates of the drug. Some investigators have attempted to solve this problem by adding other materials to the formulation during milling process to either reduce agglomeration, increase dissolution, or both. Unfortunately, this only exacerbates the uniformity and homogeneity problems discussed above because of the addition of more material relative to the level of drug and the variability of different materials to fracture during milling causing a wide range of particle size distribution. For example, when starch was added to alvimopan and the formulation was micronized, the formulation had a mean particle size diameter of about 10 microns with some particles have a particle size diameter as high as about 200 microns. This wide variation in particle size distribution could lead to problems with uniformity.

What would be desirable are compositions containing alvimopan or related 4-aryl substituted piperidine compounds that are zwitterionic in nature that could be formed into solid dosage forms where the drug is uniformly distributed, achieves the desired bioavailability, and is stable. The present invention is directed to these and other important objectives.

### SUMMARY OF THE INVENTION

In one embodiment, the present invention is directed to methods, comprising the steps of:
a. providing a composition, comprising:
   (i) at least one compound of formula I or a pharmaceutically acceptable salt or stable polymorph thereof: 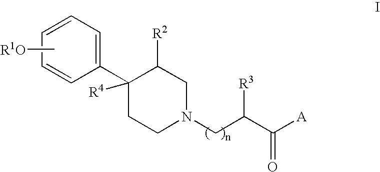
   wherein:
      R¹ is hydrogen or alkyl;
      R² is hydrogen, alkyl or alkenyl;
      R³ is hydrogen, alkyl, alkenyl, aryl, cycloalkyl, cycloalkenyl, cycloalkyl-substituted alkyl, cycloalkenyl-substituted alkyl or aralkyl;
      R⁴ is hydrogen, alkyl or alkenyl;
      A is OR⁵ or NR⁶R⁷;
      R⁵ is hydrogen, alkyl, alkenyl, cycloalkyl, cycloalkenyl, cycloalkyl-substituted alkyl, cycloalkenyl-substituted alkyl, or aralkyl;
      R⁶ is hydrogen or alkyl;
      R⁷ is hydrogen, alkyl, alkenyl, cycloalkyl, aryl, cycloalkyl-substituted alkyl, cycloalkenyl, cycloalkenyl-substituted alkyl, aralkyl, aralkyl, or alkylene substituted B or, together with the nitrogen atom to which they are attached, R⁶ and R⁷ form a heterocyclic ring;
      B is 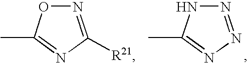
      C(═O)W or NR⁸R⁹;
      R⁸ is hydrogen or alkyl;
      R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
      W is OR¹⁰, NR¹¹R¹², or OE;
      R¹⁰ is hydrogen, alkyl, alkenyl, cycloalkyl, cycloalkenyl, cycloalkyl-substituted alkyl, cycloalkenyl-substituted alkyl, or aralkyl;
      R¹¹ is hydrogen or alkyl;
      R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
      E is 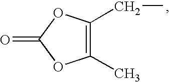
      alkylene substituted (C═O)D, or —R¹³OC(═O)R¹⁴;
      R¹³ is alkyl substituted alkylene;
      R¹⁴ is alkyl;
      D is OR⁵ or NR¹⁶R⁷;
      R¹⁵ is hydrogen, alkyl, alkenyl, cycloalkyl, cycloalkenyl, cycloalkyl-substituted alkyl, cycloalkenyl-substituted alkyl, or aralkyl;
      R¹⁶ is hydrogen, alkyl, alkenyl, aryl, aralkyl, cycloalkyl, cycloalkenyl, cycloalkyl-substituted alkyl or cycloalkenyl-substituted alkyl;
      R¹⁷ is hydrogen or alkyl or, together with the nitrogen atom to which they are attached, R¹⁶ and R¹⁷ form a heterocyclic ring;
      Y is OR¹⁸ or NR¹⁹R²⁰;
      R¹⁸ is hydrogen, alkyl, alkenyl, cycloalkyl, cycloalkenyl, cycloalkyl-substituted alkyl, cycloalkenyl-substituted alkyl, or aralkyl;
      R¹⁹ is hydrogen or alkyl;
      R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
      R²¹ is hydrogen or alkyl; and
      n is 0 to 4; and
   (ii) a pharmaceutically-acceptable excipient selected from the group consisting of mannitol, dextrose, fructose, lactose, sucrose, dextrate, maltodextrin, and mixtures thereof; and
b. micronizing said composition.

In further embodiments, the invention is directed to products produced by the methods of described above.

In yet further embodiments, the invention is directed to compositions, comprising:
a. at least one compound of formula I or a pharmaceutically acceptable salt or stable polymorph thereof: 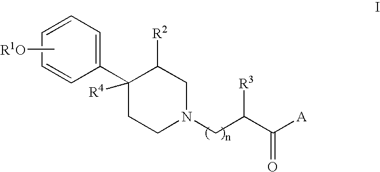
wherein:
   R¹ is hydrogen or alkyl;
   R² is hydrogen, alkyl, or alkenyl;
   R³ is hydrogen, alkyl, alkenyl, aryl, cycloalkyl, cycloalkenyl, cycloalkyl-substituted alkyl, cycloalkenyl-substituted alkyl, or aralkyl;
   R⁴ is hydrogen, alkyl, or alkenyl;
   A is OR⁵ or NR⁶R⁷;
   R⁵ is hydrogen, alkyl, alkenyl, cycloalkyl, cycloalkenyl, cycloalkyl-substituted alkyl, cycloalkenyl-substituted alkyl, or aralkyl;
   R⁶ is hydrogen or alkyl;
   R⁷ is hydrogen, alkyl, alkenyl, cycloalkyl, aryl, cycloalkyl-substituted alkyl, cycloalkenyl, cycloalkenyl-substituted alkyl, aralkyl, aralkyl, or alkylene substituted B or, together with the nitrogen atom to which they are attached, R⁶ and R⁷ form a heterocyclic ring;
   B is 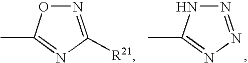 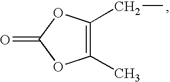
   C(═O)W or NR⁸R⁹;
   R⁸ is hydrogen or alkyl;
   R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
   W is OR¹⁰, NR¹¹R¹²,or OE;
   R¹⁰ is hydrogen, alkyl, alkenyl, cycloalkyl, cycloalkenyl, cycloalkyl-substituted alkyl, cycloalkenyl-substituted alkyl, or aralkyl;
   R¹¹ is hydrogen or alkyl;
   R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
   E is
   alkylene substituted (C═O)D, or —R¹³OC(═O)R¹⁴;
   R¹³ is alkyl-substituted alkylene;
   R¹⁴ is alkyl;
   D is OR¹⁵ or NR¹⁶R¹⁷;
   R¹⁵ is hydrogen, alkyl, alkenyl, cycloalkyl, cycloalkenyl, cycloalkyl-substituted alkyl, cycloalkenyl-substituted alkyl, or aralkyl;
   R¹⁶ is hydrogen, alkyl, alkenyl, aryl, aralkyl, cycloalkyl, cycloalkenyl, cycloalkyl-substituted alkyl, or cycloalkenyl-substituted alkyl;
   R¹⁷ is hydrogen or alkyl or, together with the nitrogen atom to which they are attached, R¹⁶ and R¹⁷ form a heterocyclic ring;
   Y is OR¹⁸ or NR¹⁹R²⁰;
   R¹⁸ is hydrogen, alkyl, alkenyl, cycloalkyl, cycloalkenyl, cycloalkyl-substituted alkyl, cycloalkenyl-substituted alkyl, or aralkyl;
   R¹⁹ is hydrogen or alkyl;
   R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
   R²¹ is hydrogen or alkyl; and
   n is 0 to 4;
(ii) a pharmaceutically-acceptable excipient selected from the group consisting of mannitol, dextrose, fructose, lactose, sucrose, dextrate, maltodextrin, and mixtures thereof; and
   wherein said composition has an average particle size range of about 5 microns to about 20 microns.

In yet other embodiments, the invention is directed to methods of preventing or treating a side effect associated with an opioid in a patient, comprising the step of:
administering to said patient in need thereof an effective amount of the above-described composition.

The methods are useful in the prevention and treatment of ileus, pruritis, constipation, urinary retention, biliary spasm, opioid bowel dysfunction, colic, nausea, or vomiting or combinations thereof, particularly postoperative ileus, postpartum ileus, opioid bowel dysfunction, postoperative nausea, or postoperative vomiting or combinations thereof.

In other embodiments, the invention is directed to methods of preventing or treating pain in a patient, comprising the step of:
administering to said patient in need thereof an effective amount of the above-described composition.
   In preferred embodiments, the composition further comprises at least one opioid.

These and other aspects of the invention will become more apparent from the following detailed description.

### DETAILED DESCRIPTION OF THE INVENTION

As employed above and throughout the disclosure, the following terms, unless otherwise indicated, shall be understood to have the following meanings.

As used herein, “oral administration” refers to the administration of a drug to a patient by way of the alimentary tract.

As used herein, “bioavailability” refers to the rate and extent to which a drug or other substance becomes available to the target tissue after administration. In the context of this invention, bioavailability refers to the degree to which the opioid antagonist becomes available to the opioid receptors in the central nervous system or peripheral thereto.

As used herein, “stable polymorph” refers to a polymorph of the compound of formula I or a pharmaceutically acceptable salt thereof that maintains its form for at least one year, preferably at least about two years, and more preferably at least about three years (time) at a temperature of about 25° C. to 30° C. and a relative humidity of about 40% to 60%.

As used herein, “micronization” or “micronizing” refers to a high-energy, dry-milling process that reduces the particle size of a material, including drugs, to ultrafine size, typically in the range of one to ten microns. The material may be micronized by interparticular impact and/or attrition, using a device such as a fluid energy mill (like a air attrition mill). This type of device uses a high velocity stream of gaseous fluid to impart a high velocity spiral movement to the material to be reduced in particle size. Typically, the gaseous fluid is introduced into the fluid energy mill at about 100 pounds per square inch. For a given material, the extent of particle size reduction depends upon a combination of gas pressure, mechanical configuration of the mill and the feed rate of the material, in addition to the fracturability of the material. Micronization using fluid energy mills is well-known in the art. See, for example, the Encyclopedia of Pharmaceutical Technology, Volume 3, page 116 (editors, James Swarbrick and James C. Boylan).

As used herein, “alkyl” refers to an optionally substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms, herein referred to as “lower alkyl”, being preferred. “Branched” refers to an alkyl group in which a lower alkyl group, such as methyl, ethyl, or propyl, is attached to a linear alkyl chain. In certain preferred embodiments, the alkyl group is a C₁-C₅ alkyl group, i.e., a branched or linear alkyl group having from 1 to about 5 carbons. In other preferred embodiments, the alkyl group is a C₁-C₃ alkyl group, i.e., a branched or linear alkyl group having from 1 to about 3 carbons. Exemplary alkyl groups include methyl, ethyl, n-propyl, isopropyl, butyl, isobutyl, sec-butyl, tert-butyl, pentyl, hexyl, heptyl, octyl, nonyl, and decyl. “Lower alkyl” refers to an alkyl group having 1 to about 6 carbon atoms. Preferred alkyl groups include the lower alkyl groups of 1 to about 3 carbons. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “alkylene” refers to a bivalent alkyl radical having the general formula —(CH₂)ₙ—, where n is 1 to 10, and all combinations and subcombinations of ranges therein. The alkylene group may be straight, branched or cyclic. Non-limiting examples include methylene, methylene (—CH₂—), ethylene (—CH₂CH₂—), propylene (—(CH₂)₃—), trimethylene, pentamethylene, and hexamethylene. There may be optionally inserted along the alkylene group one or more oxygen, sulfur or optionally substituted nitrogen atoms, wherein the nitrogen substituent is alkyl as described previously. Alkylene groups can be optionally substituted. The term “lower alkylene” herein refers to those alkylene groups having from about 1 to about 6 carbon atoms. Preferred alkylene groups have from about 1 to about 4 carbons.

As used herein, “alkenyl” refers to a monovalent alkyl radical containing at least one carbon-carbon double bond and having from 2 to about 10 carbon atoms in the chain, and all combinations and subcombinations of ranges therein. Alkenyl groups can be optionally substituted. In certain preferred embodiments, the alkenyl group is a C₂-C₁₀ alkyl group, i.e., a branched or linear alkenyl group having from 2 to about 10 carbons. In other preferred embodiments, the alkenyl group is a C₂-C₆ alkenyl group, i.e., a branched or linear alkenyl group having from 2 to about 6 carbons. In still other preferred embodiments, the alkenyl group is a C₃-C₁₀ alkenyl group, i.e., a branched or linear alkenyl group having from about 3 to about 10 carbons. In yet other preferred embodiments, the alkenyl group is a C₂-C₅ alkenyl group, i.e., a branched or linear alkenyl group having from 2 to about 5 carbons. Exemplary alkenyl groups include, for example, vinyl, propenyl, butenyl, pentenyl hexenyl, heptenyl, octenyl, nonenyl and decenyl groups.

As used herein, “aryl” refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

As used herein, “aralkyl” refers to alkyl radicals bearing an aryl substituent and have from about 6 to about 50 carbon atoms (and all combinations and subcombinations of ranges and specific numbers of carbon atoms therein), with from about 6 to about 10 carbon atoms being preferred. Aralkyl groups can be optionally substituted in either the aryl or alkyl portions. Non-limiting examples include, for example, phenylmethyl (benzyl), diphenylmethyl, triphenylmethyl, phenylethyl, diphenylethyl and 3-(4-methylphenyl)propyl.

As used herein, “heteroaryl” refers to an optionally substituted, mono-, di-, tri-, or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

As used herein, “cycloalkyl” refers to an optionally substituted, alkyl group having one or more rings in their structures having from about 3 to about 20 carbon atoms (and all combinations and subcombinations of ranges and specific numbers of carbon atoms therein), with from about 3 to about 10 carbon atoms being preferred, with from about 3 to about 8 carbon atoms being more preferred, with from about 3 to about 6 carbon atoms being even more preferred. Multi-ring structures may be bridged or fused ring structures. The cycloalkyl group may be optionally substituted with, for example, alkyl, preferably C₁-C₃ alkyl, alkoxy, preferably C₁-C₃ alkoxy, or halo. Non-limiting examples include, for example, cyclopropyl, cyclobutyl, cyclopentyl, cyclohexyl, cycloheptyl cyclooctyl, and adamantyl.

As used herein, “cycloalkyl-substituted alkyl” refers to a linear alkyl group, preferably a lower alkyl group, substituted at a terminal carbon with a cycloalkyl group, preferably a C₃-C₈ cycloalkyl group. Non-limiting examples include, for example, cyclohexylmethyl, cyclohexylethyl, cyclopentylethyl, cyclopentylpropyl, cyclopropylmethyl, and the like.

As used herein, “cycloalkenyl” refers to an olefinically unsaturated cycloalkyl group having from about 4 to about 10 carbons, and all combinations and subcombinations of ranges therein. In preferred embodiments, the cycloalkenyl group is a C₅-C₈ cycloalkenyl group, i.e., a cycloalkenyl group having from about 5 to about 8 carbons.

As used herein, “alkylcycloalkyl” refers to an optionally substituted ring system comprising a cycloalkyl group having one or more alkyl substituents. Non-limiting examples include, for example, alkylcycloalkyl groups include 2-methylcyclohexyl, 3,3-dimethylcyclopentyl, trans-2,3-dimethylcyclooctyl, and 4-methyldecahydronaphthalenyl.

As used herein, “heteroaralkyl” refers to an optionally substituted, heteroaryl substituted alkyl radicals having from about 2 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

As used herein, “heterocycloalkyl” refers to an optionally substituted, mono-, di-, tri-, or other multicyclic aliphatic ring system that includes at least one, and preferably from 1 to about 4 sulfur, oxygen, or nitrogen heteroatom ring members. Heterocycloalkyl groups can have from about 3 to about 20 carbon atoms (and all combinations and subcombinations of ranges and specific numbers of carbon atoms therein), with from about 4 to about 10 carbons being preferred. The heterocycloalkyl group may be unsaturated, and may also be fused to aromatic rings. Non-limiting examples include, for example, tetrahydrofuranyl, tetrahydrothienyl, piperidinyl, pyrrolidinyl, isoxazolidinyl, isothiazolidinyl, pyrazolidinyl, oxazolidinyl, thiazolidinyl, piperazinyl, morpholinyl, piperadinyl, decahydroquinolyl, octahydrochromenyl, octahydro-cyclopenta[c]pyranyl, 1,2,3,4,-tetrahydroquinolyl, octahydro-[2]pyrindinyl, decahydro-cycloocta[c]furanyl, and imidazolidinyl.

As used herein, the term “spiroalkyl” refers to an optionally substituted, alkylene diradical, both ends of which are bonded to the same carbon atom of the parent group to form a spirocyclic group. The spiroalkyl group, taken together with its parent group, as herein defined, has 3 to 20 ring atoms. Preferably, it has 3 to 10 ring atoms. Non-limiting examples of a spiroalkyl group taken together with its parent group include 1-(1-methyl-cyclopropyl)-propan-2-one, 2-(1-phenoxy-cyclopropyl)-ethylamine, and 1-methyl-spiro[4.7]dodecane.

As used herein, the term “alkoxy” refers to an optionally substituted alkyl-O— group wherein alkyl is as previously defined. Non-limiting examples include, for example, include methoxy, ethoxy, n-propoxy, i-propoxy, n-butoxy, and heptoxy.

As used herein, the term “aryloxy” refers to an optionally substituted aryl-O— group wherein aryl is as previously defined. Non-limiting examples include, for example, phenoxy and naphthoxy.

As used herein, the term “aralkoxy” refers to an optionally substituted aralkyl-O— group wherein aralkyl is as previously defined. Non-limiting examples include, for example, benzyloxy, 1-phenylethoxy, 2-phenylethoxy, and 3-naphthylheptoxy.

As used herein, the term “aryloxyaryl” refers to an aryl group with an aryloxy substituent wherein aryloxy and aryl are as previously defined. Aryloxyaryl groups can be optionally substituted. Non-limiting examples include, for example, phenoxyphenyl, and naphthoxyphenyl.

As used herein, the term “heteroarylaryl” refers to an aryl group with a heteroaryl substituent wherein heteroaryl and aryl are as previously defined. Heteroarylaryl groups can be optionally substituted. Non-limiting examples include, for example, 3-pyridylphenyl, 2-quinolylnaphthalenyl, and 2-pyrrolylphenyl.

As used herein, the term “alkoxyaryl” refers to an aryl group bearing an alkoxy substituent wherein alkoxy and aryl are as previously defined. Alkoxyaryl groups can be optionally substituted. Non-limiting examples include, for example, para-anisyl, meta-t-butoxyphenyl, and methylendioxyphenyl.

As used herein, “carboxy” refers to a —C(═O)OH group.

As used herein, “alkanoyl” refers to a —C(═O)-alkyl group, wherein alkyl is as previously defined. Exemplary alkanoyl groups include acetyl (ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

As used herein, “heterocyclic” refers to a monocyclic or multicyclic ring system carbocyclic radical containing from about 4 to about 10 members, and all combinations and subcombinations of ranges therein, wherein one or more of the members is an element other than carbon, for example, nitrogen, oxygen or sulfur. The heterocyclic group may be aromatic or nonaromatic. Non-limiting examples include, for example, pyrrole and piperidine groups.

As used herein, “halo” refers to fluoro, chloro, or bromo.

Typically, substituted chemical moieties include one or more substituents that replace hydrogen. Exemplary substituents include, for example, halo (e.g., F, Cl, Br, I), alkyl, cycloalkyl, alkylcycloalkyl, alkenyl, alkynyl, aralkyl, aryl, heteroaryl, heteroaralkyl, spiroalkyl, heterocycloalkyl, hydroxyl (—OH), nitro (—NO₂), cyano (—CN), amino (—NH₂), —N-substituted amino (—NHR″), —N,N-disubstituted amino (—N(R″)R″), carboxyl (—COOH), —C(═O)R″, —OR″, —C(═O)OR″, —NHC(═O)R″, aminocarbonyl (—C(═O)NH₂), —N-substituted aminocarbonyl (—C(═O)NHR″), —N,N-disubstituted aminocarbonyl (—C(═O)N(R″)R″), thiol, thiolato (SR″), sulfonic acid (SO₃H), phosphonic acid (PO₃H), S(═O)₂R″, S(═O)₂NH₂, S(═O)₂ NHR″, S(═O)₂NR″R″, NHS(═O)₂R″, NR″S(═O)₂R″, CF₃, CF₂CF₃, NHC(═O)NHR″, NHC(═O)NR″R″, NR″C(═O)NHR″, NR″C(═O)NR″R″, NR″C(═O)R″ and the like. In relation to the aforementioned substituents, each moiety R″ can be, independently, any of H, alkyl, cycloalkyl, alkenyl, aryl, aralkyl, heteroaryl, or heterocycloalkyl, for example.

As used herein, “side effect” refers to a consequence other than the one(s) for which an agent or measure is used, as the adverse effects produced by a drug, especially on a tissue or organ system other then the one sought to be benefited by its administration. In the case, for example, of opioids, the term “side effect” may refer to such conditions as, for example, ileus, pruritis, constipation, urinary retention, biliary spasm, opioid bowel dysfunction, colic, nausea, or vomiting or a combination thereof.

As used herein, “ileus” refers to the obstruction of the bowel or gut, especially the colon. See, e.g., Dorland's Illustrated Medical Dictionary, p. 816, 27th ed. (W.B. Saunders Company, Philadelphia 1988). Ileus should be distinguished from constipation, which refers to infrequent or difficulty in evacuating the feces. See, e.g., Dorland's Illustrated Medical Dictionary, p. 375, 27th ed. (W.B. Saunders Company, Philadelphia 1988). Ileus may be diagnosed by the disruption of normal coordinated movements of the gut, resulting in failure of the propulsion of intestinal contents. See, e.g., Resnick, J. Am. J. of Gastroenterology, 1992, 751 and Resnick, J. Am. J. of Gastroenterology, 1997, 92, 934. In some instances, particularly following surgery, including surgery of the abdomen, the bowel dysfunction may become quite severe, lasting for more than a week and affecting more than one portion of the gastrointestinal tract. This condition is often referred to as postsurgical (or postoperative) ileus and most frequently occurs after laparotomy (see Livingston, E. H. and Passaro, E. D. Jr., Digestive Diseases and Sciences, 1990, 35, 121). Similarly, postpartum ileus is a common problem for women in the period following childbirth, and is thought to be caused by similar fluctuations in natural opioid levels as a result of birthing stress.

As used herein, “effective amount” refers to an amount of a compound as described herein that may be therapeutically effective to inhibit, prevent, or treat the symptoms of particular disease, disorder, or side effect. Such diseases, disorders and side effects include, but are not limited to, those pathological conditions associated with the administration of opioids (for example, in connection with the treatment and/or prevention of pain), wherein the treatment or prevention comprises, for example, inhibiting the activity thereof by contacting cells, tissues or receptors with compounds of the present invention. Thus, for example, the term “effective amount,” when used in connection with opioids, for example, for the treatment of pain, refers to the treatment and/or prevention of the painful condition. The term “effective amount,” when used in connection with peripheral μ opioid antagonists, refers to the treatment and/or prevention of side effects typically associated with opioids including, for example, such side effects as ileus, pruritis, constipation, urinary retention, biliary spasm, opioid bowel dysfunction, colic, nausea, or vomiting or a combination thereof.

As used herein, “in combination with,” “combination therapy” and “combination products” refer, in certain embodiments, to the concurrent administration to a patient of antiemetic agents and peripheral μ opioid antagonists, including, for example, the compounds of formula I, or to the concurrent administration to a patient of antiemetic agents, peripheral μ opioid antagonists, and opioids. When administered in combination, each component may be administered at the same time or sequentially in any order at different points in time. Thus, each component may be administered separately but sufficiently closely in time so as to provide the desired therapeutic effect.

As used herein, “dosage unit” refers to physically discrete units suited as unitary dosages for the particular patient to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable metal salt” refer to derivatives of the disclosed compounds wherein the parent compound is modified by making base salts thereof. Examples of pharmaceutically acceptable salts include, but are not limited to, mineral or organic acid salts of basic residues such as amines, alkali and the like. The pharmaceutically acceptable salts include the conventional non-toxic salts of the parent compound formed, for example, from non-toxic inorganic or organic bases. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein throughout, can be used or prepared in alternate forms. Isomorphic crystalline forms, all chiral and racemic forms, N-oxide, hydrates, and solvates are also contemplated to be within the scope of the present invention.

Certain acidic or basic compounds of the present invention may exist as zwitterions. All forms of the compounds, including free acid, free-base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

As used herein, “patient” refers to animals, including mammals, preferably humans.

As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “stereoisomers” refers to compounds that have identical chemical constitution, but differ as regards the arrangement of the atoms or groups in space.

As used herein, “N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

When any variable occurs more than one time in any constituent or in any formula, its definition in each occurrence is independent of its definition at every other occurrence. Combinations of substituents and/or variables are permissible only if such combinations result in stable compounds.

The piperidines derivatives useful in the methods and compositions of the invention as illustrated in formula I can occur as the trans and cis stereochemical isomers at the 3- and 4-positions of the piperidine ring. In the most preferred compounds of formula I, the R² substituent and the R⁴ substituent are in the “trans” orientation on the piperidine.

In addition to the “cis” and trans” orientation of the R² substituent and the R⁴ substituent of formula I, the absolute stereochemistry of the carbon atoms bearing R² substituent and the R⁴ substituent of formula I is also defined as using the commonly employed “R” and “S” definitions (Orchin et al., The Vocabulary of Organic Chemistry, John Wiley and Sons, Inc., page 126, which is incorporated herein by reference). The preferred compounds of the present invention are those in which the configuration of both the R² substituent and the R⁴ substituents of formula I on the piperidine ring are “R.”

Furthermore, asymmetric carbon atoms may be introduced into the molecule depending on the structure of R⁴. As such, these classes of compounds can exist as the individual “R” or “S” stereoisomers at these chiral centers, or the racemic mixture of the isomers, and all are contemplated as within the scope of the present invention. Preferably, a substantially pure stereoisomer of the compounds of this invention is used, i.e., an isomer in which the configuration at the chiral center is “R” or “S”, i.e., those compounds in which the configuration at the three chiral centers I preferably 3R, 4R, S or 3R, 4R, R.

As used herein, “peripheral” or “peripherally-acting” refers to an agent that acts outside of the central nervous system.

As used herein, “centrally-acting” refers to an agent that acts within the central nervous system.

The methods and compositions of the present invention involve a peripheral opioid antagonist compound. The term “peripheral” designates that the compound acts primarily on physiological systems and components external to the central nervous system. In preferred form, the peripheral opioid antagonist compounds employed in the methods of the present invention exhibit high levels of activity with respect to peripheral tissue, such as, gastrointestinal tissue, while exhibiting reduced, and preferably substantially no, CNS activity. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably less than about 1% of the pharmacological activity of the compounds employed in the present methods is exhibited in the CNS.

Furthermore, it is preferred in certain embodiments of the invention where the compound is administered to antagonize the peripheral side effects of an opioid that the compound does not substantially cross the blood-brain barrier and thereby decrease the beneficial activity of the opioid. The phrase “does not substantially cross,” as used herein, means that less than about 20% by weight of the compound employed in the present methods crosses the blood-brain barrier, preferably less than about 15% by weight, more preferably less than about 10% by weight, even more preferably less than about 5% by weight and most preferably 0% by weight of the compound crosses the blood-brain barrier. Selected compounds can be evaluated for CNS penetration by determining plasma and brain levels following intravenous administration.

U.S. Pat. No. 6,45 1,806 and U.S. Pat. No. 6,469,030 disclose methods and compositions comprising opioids and opioid antagonists, including peripheral μ opioid antagonists, the disclosures of which are incorporated herein by reference in their entirety. The methods and compositions are useful, inter alia, for treating and/or preventing pain and for treating and/or preventing side effects associated with opioids including ileus, pruritis, constipation, urinary retention, biliary spasm, opioid bowel dysfunction, colic, vomiting or nausea or a combination thereof, particularly postoperative or postpartum ileus, opioid bowel dysfunction, postoperative nausea, or postoperative vomiting. The methods and compositions of the present invention are related to peripheral μ opioid antagonists and are directed to combinations of peripheral t opioid antagonists with centrally-acting antiemetic agents and with centrally-acting antiemetic agents and opioids, for the treatment and prevention, for example, of pain and/or side effects associated with opioids, including ileus, pruritis, constipation, urinary retention, biliary spasm, opioid bowel dysfunction, colic, vomiting or nausea or a combination thereof, particularly postoperative or postpartum ileus, opioid bowel dysfunction, postoperative nausea, or postoperative vomiting.

The methods of the present invention are useful, inter alia, for forming compositions, especially solid dosage forms, where the drug is uniformly distributed, achieves the desired bioavailability, and is stable, relative to prior art compositions.

Accordingly, in one embodiment, the present invention provides methods comprising the steps of:
a. providing a composition, comprising:
   (i) at least one compound of formula I or a pharmaceutically acceptable salt or stable polymorph thereof: 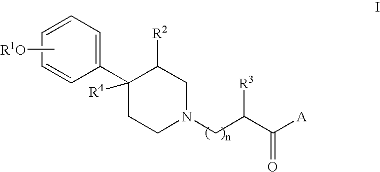
   wherein:
      R¹ is hydrogen or alkyl;
      R² is hydrogen, alkyl or alkenyl;
      R³ is hydrogen, alkyl, alkenyl, aryl, cycloalkyl, cycloalkenyl, cycloalkyl-substituted alkyl, cycloalkenyl-substituted alkyl or aralkyl;
      R⁴ is hydrogen, alkyl or alkenyl;
      A is OR⁵ or NR⁶R⁷;
      R⁵ is hydrogen, alkyl, alkenyl, cycloalkyl, cycloalkenyl, cycloalkyl-substituted alkyl, cycloalkenyl-substituted alkyl, or aralkyl;
      R⁶ is hydrogen or alkyl;
      R⁷ is hydrogen, alkyl, alkenyl, cycloalkyl, aryl, cycloalkyl-substituted alkyl, cycloalkenyl, cycloalkenyl-substituted alkyl, aralkyl, aralkyl, or alkylene substituted B or, together with the nitrogen atom to which they are attached, R⁶ and R⁷ form a heterocyclic ring;
      B is 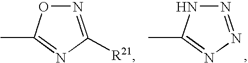
      C(═O)W or NR⁸R⁹;
      R⁸ is hydrogen or alkyl;
      R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
      W is OR¹⁰, NR¹¹R¹², or OE;
      R¹⁰ is hydrogen, alkyl, alkenyl, cycloalkyl, cycloalkenyl, cycloalkyl-substituted alkyl, cycloalkenyl-substituted alkyl, or aralkyl;
      R¹¹ is hydrogen or alkyl;
      R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
      E is 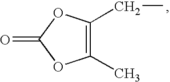
      alkylene substituted (C═O)D, or —R¹³OC(═O)R¹⁴;
      R¹³ is alkyl substituted alkylene;
      R¹⁴ is alkyl;
      D is OR¹⁵ or NR¹⁶R¹⁷;
      R¹⁵ is hydrogen, alkyl, alkenyl, cycloalkyl, cycloalkenyl, cycloalkyl-substituted alkyl, cycloalkenyl-substituted alkyl, or aralkyl;
      R¹⁶ is hydrogen, alkyl, alkenyl, aryl, aralkyl, cycloalkyl, cycloalkenyl, cycloalkyl-substituted alkyl or cycloalkenyl-substituted alkyl;
      R¹⁷ is hydrogen or alkyl or, together with the nitrogen atom to which they are attached, R¹⁶ and R¹⁷ form a heterocyclic ring;
      Y is OR¹⁸ or NR¹⁹R²⁰;
      R¹⁸ is hydrogen, alkyl, alkenyl, cycloalkyl, cycloalkenyl, cycloalkyl-substituted alkyl, cycloalkenyl-substituted alkyl, or aralkyl;
      R¹⁹ is hydrogen or alkyl;
      R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
      R²¹ is hydrogen or alkyl; and
      n is 0 to 4; and
   (ii) a pharmaceutically-acceptable excipient selected from the group consisting of mannitol, dextrose, fructose, lactose, sucrose, dextrate, maltodextrin, and mixtures thereof; and
b. micronizing said composition.

In certain embodiments, the invention is directed to compositions, comprising:
a. at least one compound of formula I or a pharmaceutically acceptable salt or stable polymorph thereof: 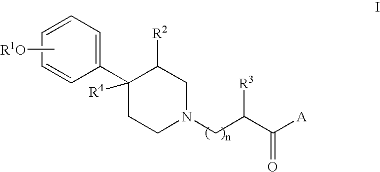 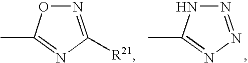
wherein:
   R¹ is hydrogen or alkyl;
   R² is hydrogen, alkyl, or alkenyl;
   R³ is hydrogen, alkyl, alkenyl, aryl, cycloalkyl, cycloalkenyl, cycloalkyl-substituted alkyl, cycloalkenyl-substituted alkyl, or aralkyl;
   R⁴ is hydrogen, alkyl, or alkenyl;
   A is OR⁵ or NR⁶R⁷;
   R⁵ is hydrogen, alkyl, alkenyl, cycloalkyl, cycloalkenyl, cycloalkyl-substituted alkyl, cycloalkenyl-substituted alkyl, or aralkyl;
   R⁶ is hydrogen or alkyl;
   R⁷ is hydrogen, alkyl, alkenyl, cycloalkyl, aryl, cycloalkyl-substituted alkyl, cycloalkenyl, cycloalkenyl-substituted alkyl, aralkyl, aralkyl, or alkylene substituted B or, together with the nitrogen atom to which they are attached, R⁶ and R⁷ form a heterocyclic ring;
   B is
   C(═O)W or NR⁸R⁹;
   R⁸ is hydrogen or alkyl;
   R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
   W is OR¹⁰, NR¹¹R¹², or OE;
   R¹⁰ is hydrogen, alkyl, alkenyl, cycloalkyl, cycloalkenyl, cycloalkyl-substituted alkyl, cycloalkenyl-substituted alkyl, or aralkyl;
   R¹¹ is hydrogen or alkyl;
   R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
   E is 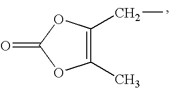
   alkylene substituted (C═O)D, or —R¹³OC(═O)R¹⁴;
   R¹³ is alkyl-substituted alkylene;
   R¹⁴ is alkyl;
   D is OR¹⁵ or NR¹⁶R¹⁷;
   R¹⁵ is hydrogen, alkyl, alkenyl, cycloalkyl, cycloalkenyl, cycloalkyl-substituted alkyl, cycloalkenyl-substituted alkyl, or aralkyl;
   R¹⁶ is hydrogen, alkyl, alkenyl, aryl, aralkyl, cycloalkyl, cycloalkenyl, cycloalkyl-substituted alkyl, or cycloalkenyl-substituted alkyl;
   R¹⁷ is hydrogen or alkyl or, together with the nitrogen atom to which they are attached, R¹⁶ and R¹⁷ form a heterocyclic ring;
   Y is OR¹⁸ or NR¹⁹R²⁰;
   R¹⁸ is hydrogen, alkyl, alkenyl, cycloalkyl, cycloalkenyl, cycloalkyl-substituted alkyl, cycloalkenyl-substituted alkyl, or aralkyl;
   R¹⁹ is hydrogen or alkyl;
   R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
   R²¹ is hydrogen or alkyl; and
   n is 0 to 4;
(b) a pharmaceutically-acceptable excipient selected from the group consisting of mannitol, dextrose, fructose, lactose, sucrose, dextrate, maltodextrin, and mixtures thereof; and
   wherein said composition has an average particle size range of about 5 microns to about 20 microns.

While not wishing to be bound by theory, it is believed that the selection of the excipients useful in the methods and compositions of the invention work because the fracturability of the select excipients, namely mannitol, dextrose, fructose, lactose, sucrose, dextrate, maltodextrin, and mixtures thereof, especially mannitol, is similar to the fracturability of the compounds of formula I, including alvimopan. It is believed that when the select mixture is milled the two or more materials (select excipient(s) and compound of formula I) fracture similarly and a uniform size distribution of particles is formed.

In preferred embodiments, the weight ratio of the compound of formula I or a pharmaceutically acceptable salt or stable polymorph thereof to the pharmaceutically acceptable excipient is about 10:1 to about 1:10, more preferably, about 5:1 to about 1:5, even more preferably, about 2:1 to about 1:2, and most preferably, about 1:1.

In preferred embodiments, the compound of formula I or a pharmaceutically acceptable salt or stable polymorph thereof has an average particle size range of about 5 microns to about 20 microns, preferably about 5 microns to about 10 microns.

In preferred embodiments, the pharmaceutically-acceptable excipient is mannitol.

In preferred embodiments, the pharmaceutically-acceptable excipient has an average particle size range of about 5 microns to about 20 microns, preferably about 5 microns to about 10 microns.

In preferred embodiments, the average particle size of said compound of formula I or a pharmaceutically acceptable salt or stable polymorph thereof differs from the average particle size of said excipient by no more than about 200%, more preferably, no more than about 100%, even more preferably, no more than about 50%, yet even more preferably, no an about 25%, and still even more preferably, no more than about 10%.

In certain preferred embodiments, the compositions of the invention may include an opioid, a prodrug of an opioid, and/or pharmacologically-active metabolites, provided that its inclusion does not interfere with the solubility or bioavailability of the compound of formula I. Preferably, the opioid has an average particle size range of about 5 microns to about 20 microns. The opioid may be incorporated into the composition of the compound of formula I and the pharmaceutically-acceptable excipient selected from the group of mannitol, dextrose, fructose, lactose, sucrose, dextrate, maltodextrin, and mixtures thereof, before the micronizing step. Alternatively or additionally, the opioid may be mixed with the micronized composition of the compound of formula I and the pharmaceutically-acceptable excipient selected from the group of mannitol, dextrose, fructose, lactose, sucrose, dextrate, maltodextrin, and mixtures thereof. Suitable opioids include alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol, and mixtures thereof. Preferred opioids include morphine, codeine, oxycodone, hydrocodone, dihydrocodeine, propoxyphene, fentanyl, and tramadol.

In certain preferred embodiments where an opioid is present, the composition of the invention is formed into a controlled release formulation, especially where the compound of formula I is available for immediate release and where the opioid is available for a delayed and/or controlled release (such as a multilayered capsule where the compound of formula I is in an immediate release layer(s) and the opioid in a delayed and/or controlled release layer(s).

Compositions of the present invention may further include one or more other active ingredients conventionally employed in analgesic and/or cough-cold-antitussive combination products, provided that its inclusion does not interfere with the solubility or bioavailability of the compound of formula I. Such conventional ingredients include, for example, aspirin, COX-2 inhibitors, acetaminophen, phenylpropanolamine, phenylephrine, chlorpheniramine, caffeine, and/or guaifenesin. Typical or conventional ingredients that may be included are described, for example, in the Physicians' Desk Reference, 2004, the disclosure of which is hereby incorporated herein by reference, in its entirety.

In addition, the composition of the invention may further include one or more compounds that may be designed to enhance the analgesic potency of the opioid and/or to reduce analgesic tolerance development, provided that its inclusion does not interfere with the solubility or bioavailability of the compound of formula I. Such compounds include, for example, dextromethorphan or other NMDA antagonists (Mao, M. J. et al., Pain 1996, 67, 361), L-364,718 and other CCK antagonists (Dourish, C. T. et al., Eur. J. Pharmacol., 1988, 147, 469), NOS inhibitors (Bhargava, H. N. et al., Neuropeptides, 1996, 30, 219), PKC inhibitors (Bilsky, E. J. et al., J. Pharmacol. Exp. Ther. 1996, 277, 484), and dynorphin antagonists or antisera (Nichols, M. L. et al., Pain, 1997, 69, 317). The disclosures of each of the foregoing documents are hereby incorporated herein by reference, in their entireties.

Other pharmaceutically acceptable excipients, opioids, and optional compounds for enhancing the analgesic potency of the opioid and/or for reducing analgesic tolerance development, that may be employed in the methods and compositions of the present invention, in addition to those exemplified above, would be readily apparent to one of ordinary skill in the art, once armed with the teachings of the present disclosure.

The manufacture of the micronized composition includes the initial blending of the pharmaceutical acceptable excipient selected from the group consisting of mannitol, dextrose, fructose, lactose, sucrose, dextrate, maltodextrin, and mixtures thereof with at least compound of formula I. Then, the composition is micronized in a device, such as an air attrition mill. This micronized blend is then filled into capsules or compressed into tablets. Alternatively, the micronized composition may be further blended with the same (mannitol, dextrose, fructose, lactose, sucrose, dextrate, maltodextrin, and mixtures thereof) or different (any suitable pharmaceutically acceptable excipient useful for solid dosage formulations) and then filled into capsules or compressed into tablets. The tablets may also film coated.

Preferred 4-aryl-piperidine derivatives include, for example, the compounds disclosed in U.S. Pat. No. 5,250,542; U.S. Pat. No. 5,159,081; U.S. Pat. No. 5,270,328; and U.S. Pat. No. 5,434,171, U.S. Pat. No. 6,451,806 and U.S. Pat. No. 6,469,030, the disclosures of which are hereby incorporated herein by reference, in their entireties.

In preferred embodiments, the compound of formula I is a trans 3,4-isomer.

In certain embodiments employing compounds of formula I, it is preferred that
R¹ is hydrogen;
R² is alkyl;
n is 1 or 2;
R³ is benzyl, phenyl, cyclohexyl, or cyclohexylmethyl; and
R⁴ is alkyl.

In certain embodiments employing compounds of formula I, it is preferred that
A is OR⁵; and
R⁵ is hydrogen or alkyl.

In certain embodiments employing compounds of formula I, it is preferred that
A is NR⁶R⁷;
R⁶ is hydrogen;
R⁷ is alkylene substituted B; and
B is C(O)W.

In certain embodiments employing compounds of formula I, it is preferred that
R⁷is (CH₂)_{q}—B;
q is about 1 to about 3;
W is OR¹⁰; and
R¹⁰ is hydrogen, alkyl, phenyl-substituted alkyl, cycloalkyl or cycloalkyl-substituted alkyl.

In certain embodiments including compounds of formula I, it is preferred that
W is NR¹¹R¹²
R¹¹ is hydrogen or alkyl; and
R¹² is hydrogen, alkyl or alkylene substituted C(═O)Y.

In certain embodiments employing compounds of formula I, it is preferred that
R¹² is (CH₂)ₘC(O)Y;
m is 1 to 3;
Y is OR¹⁸ or NR¹⁹R²⁰; and
R¹⁸, R¹⁹ and R²⁰ are independently hydrogen or alkyl.

In certain embodiments employing compounds of formula I, it is preferred that
W is OE;
E is CH₂C(═O)D;
D is OR¹⁵ or NR¹⁶R¹⁷;
R¹⁵ is hydrogen or alkyl;
R¹⁶ is methyl or benzyl; and
R¹⁷ is hydrogen.

In certain embodiments employing compounds of formula I, it is preferred that
W is OE;
E is R¹³OC(═O)R¹⁴;
R¹³ is —CH(CH₃)— or —CH(CH₂CH₃)—; and

R¹⁴ is alkyl.

In certain embodiments employing compounds of formula I, it is preferred that
A is OR⁵; and
R⁵ is hydrogen.

In certain embodiments employing compounds of formula I, it is preferred that the configuration at positions 3 and 4 of the piperidine ring is each R.

Preferred compounds of formula I include:
Q-CH₂CH(CH₂(C₆H₅))C(O)OH,
Q-CH₂CH₂CH(C₆H₅)C(O)NHCH₂C(O)OCH₂CH₂,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NHCH₂C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OCH₂CH₃,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NHCH₂C(O)OCH₂CH(CH₃)₂,
Z-NH(CH₂)₂C(O)OCH₂(C₆H₅),
Z-NH(CH₂)₂C(O)OH,
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)O(CH2)₄CH₃,
Z-NHCH₂C(O)OCH₂C(O)NHCH₃,
Z-NHCH₂C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
wherein:

Q represents

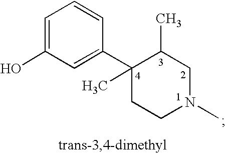

G represents

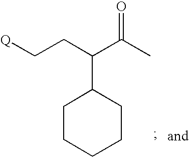

Z represents

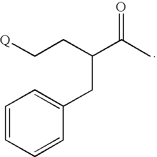

More preferred compounds of formula I include:
(3R,4R,S)—Z-NHCH₂C(O)OCH₂CH(CH₃)₂,
(+)—Z-NHCH₂C(O)OH,
(−)—Z-NHCH₂C(O)OH,
(3R,4R,R)—Z-NHCH₂C(O)—OCH₂CH(CH₃)₂,
(3S,4S,S)—Z-NHCH₂C(O)OCH₂CH(CH₃)₂,
(3S,4S,R)—Z-NHCH₂C(O)OCH₂CH(CH₃)₂,
(3R,4R)—Z-NHCH₂C(O)NHCH₂(C₆H₅) and
(3R,4R)-G-NH(CH₂)₃C(O)OH.
wherein Q, Z and G are as defined above.

Even more preferred compounds of formula I include (+)—Z-NHCH₂C(O)OH and (−)—Z-NHCH₂C(O)OH, wherein Z is as defined above. It is especially preferred when said compound is (+)—Z-NHCH₂C(O)OH. [[2(S)-[[4(R)-(3-hydroxyphenyl)-3(R),4-dimethyl-piperidinyl]methyl]-1-oxo-3-phenylpropyl]amino]acetic acid dihydrate (USAN name alvimopan) is an especially preferred compound.

Even more preferred compounds of formula I include Q-CH₂CH(CH₂(C₆H₅))C(O)OH, wherein Q is as defined above. It is especially preferred when said compound is (3R, 4R, S)-Q-CH₂CH(CH₂(C₆H₅))C(O)OH. This compound is an active metabolite of alvimopan but, when administered orally, has a much greater propensity for undesirably reversing analgesia than alvimopan. When administered parenterally, especially intraveneously, it may be administered at much lower doses with an attendant reduction in this propensity.

Compounds of formula I that act locally on the gut, have high potency, and are orally active are particularly preferred. A particularly preferred embodiment of the present invention is the compound (+)—Z-NHCH₂C(O)OH, i.e., the compound of the following formula (II):

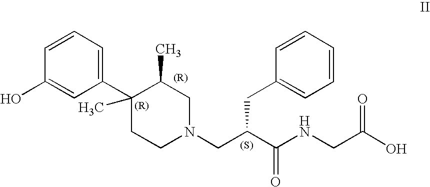

The compound of formula (II) has low solubility in water except at low or high pH conditions. Zwitterionic character may be inherent to the compound, and may impart desirable properties such as poor systemic absorption and sustained local effect on the gut following oral administration.

In especially preferred embodiments, the compound of a formula I is a substantially pure stereoisomer.

In yet other embodiments, the invention is directed to methods of preventing or treating a side effect associated with an opioid in a patient, comprising the step of:
administering to said patient in need thereof an effective amount of the above-d described composition.

The methods are useful in the prevention and treatment of ileus, pruritis, constipation, urinary retention, biliary spasm, opioid bowel dysfunction, colic, vomiting or nausea or a combination thereof, particularly postoperative or postpartum ileus, opioid bowel dysfunction, postoperative nausea, or postoperative vomiting.

In other embodiments, the invention is directed to methods of preventing or treating pain in a patient, comprising the step of:
administering to said patient in need thereof an effective amount of the above-d described composition.
   In preferred embodiments, the composition further comprises at least one opioid.

The present invention is directed to methods and compositions involving opioid compounds. As discussed above, such opioid compounds may be useful, for example, in the treatment and/or prevention of pain. However, as also discussed above, undesirable side effects including, for example, ileus, pruritis, constipation, urinary retention, biliary spasm, opioid bowel dysfunction, colic, vomiting or nausea or a combination thereof, especially postoperative and postpartum ileus, opioid bowel dysfunction, nausea and/or vomiting, as well as other side effects, may frequently occur in patients receiving opioid compounds. By virtue of the methods and compositions of the present invention, effective and desirable inhibition of undesirable side effects that may be associated with opioid compounds may be advantageously achieved. Accordingly, combination methods and compositions, where opioids are combined or co-administered with suitable peripheral μ opioid antagonist compounds, may afford an efficacy advantage over the compounds and agents alone.

In this connection, as discussed above, patients are often administered opioids for the treatment, for example, of painful conditions. However, as noted above, undesirable side effects such as, for example, ileus, pruritis, constipation, urinary retention, biliary spasm, opioid bowel dysfunction, colic, vomiting, or nausea or a combination thereof, may result from opioid administration. These undesirable side effects may act as a limiting factor in connection with the amount of opioid that may be administered to the patient. That is, the amount of opioid capable of being administered to the patient may be limited due to the undesired occurrence of the aforementioned side effects. The limited amounts of opioid that may be administered to a patient may, in turn, result in a disadvantageously diminished degree of pain alleviation. The present combination methods and compositions may be used to advantageously increase the amount of opioid administered to a patient, thereby obtaining enhanced pain alleviation, while reducing, minimizing and/or avoiding undesirable side effects that may be associated with the opioid. The peripheral μ opioid antagonists employed in the methods and compositions of the present invention preferably have substantially no central nervous system activity and, accordingly, desirably do not affect the pain killing efficacy of the opioid.

While not intending to be bound by any theory or theories of operation, it is contemplated that opioid side effects, such as ileus, pruritis, constipation, urinary retention, biliary spasm, opioid bowel dysfunction, colic, vomiting or nausea or a combination thereof, may result from undesirable interaction of the opioid with peripheral μ receptors. Administration of a peripherally-acting μ opioid antagonist according to the methods of the present invention may block interaction of the opioid compounds with the μ receptors, thereby preventing and/or inhibiting the side effects, in particular postoperative or postpartum ileus, opioid bowel dysfunction, nausea and/or vomiting.

Other μ opioid antagonist compounds that may be employed in the methods and compositions of the present invention, in addition to those exemplified above, would be readily apparent to one of ordinary skill in the art, once armed with the teachings of the present disclosure.

The compounds employed in the methods of the present invention may exist in prodrug form. As used herein, “prodrug” is intended to include any covalently bonded carriers that release the active parent drug, for example, as according to formulas I, employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example formula I, may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

The compounds employed in the methods of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As discussed in detail above, compounds employed in the present methods may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include the benzyloxycarbonyl group and the tert-butyloxycarbonyl group. Other preferred protecting groups that may be employed in accordance with the present invention may be described in Greene, T. W. and Wuts, P. G. M., Protective Groups in Organic Synthesis 2d. Ed., Wiley & Sons, 1991.

The 4-aryl-piperidine derivatives of formula I of the present invention may be synthesized employing methods taught, for example, in U.S. Pat. No. 5,250,542, U.S. Pat. No. 5,434,171, U.S. Pat. No. 5,159,081, U.S. Pat. No. 5,270,328, U.S. Pat. No. 6,451,806, U.S. Pat. No. 6,469,030, and Werner, J. A., et al., Journal of Organic Chemistry, 61, 587-597 (1996), the disclosures of which are hereby incorporated herein by reference in their entireties. For example, the 3-substituted-4-methyl-4-(3-hydroxy- or alkanoyloxyphenyl)piperidine derivatives employed as starting materials in the synthesis of the present compounds may be prepared by the general procedure taught in U.S. Pat. No. 4,115,400 and U.S. Pat. No. 4,891,379, the disclosures of which are hereby incorporated herein by reference in their entireties. The starting material for the synthesis of compounds described herein, (3R,4R)-4-(3-hydroxypheny)-3,4-dimethylpiperidine, may be prepared by the procedures described in U.S. Pat. No. 4,581,456 and U.S. Pat. No. 5,136,040, the disclosures of which are hereby incorporated herein by reference, in their entirety, but adjusted as described such that the β-stereochemistry is preferred.

The first step of the process may involve the formation of the 3-alkoxyphenyllithium reagent by reacting 3-alkoxybromobenzene with an alkyllithium reagent. This reaction may be performed under inert conditions and in the presence of a suitable non-reactive solvent such as dry diethyl ether or preferably dry tetrahydrofuran. Preferred alkyllithium reagents used in this process are n-butyl lithium, and especially sec-butyl lithium. Generally, approximately an equimolar to slight excess of alkyllithium reagent may be added to the reaction mixture. The reaction may be conducted at a temperature of from about −20° C. and about −100° C., more preferably from about −50° C. to about −55° C.

Once the 3-alkoxyphenyllithium reagent has formed, approximately an equimolar quantity of a 1-alkyl-4-piperidone may be added to the mixture while maintaining the temperature between −20° C. and −100° C. The reaction is typically complete after about 1 to 24 hours. At this point, the reaction mixture may be allowed to gradually warm to room temperature. The product may be isolated by the addition to the reaction mixture of a saturated sodium chloride solution to quench any residual lithium reagent. The organic layer may be separated and further purified if desired to provide the appropriate 1-alkyl-4-(3-alkoxyphenyl)piperidinol derivative.

The dehydration of the 4-phenylpiperidinol prepared above may be accomplished with a strong acid according to well known procedures. While dehydration occurs in various amounts with any one of several strong acids such as hydrochloric acid, hydrobromic acid, and the like, dehydration is preferably conducted with phosphoric acid, or especially p-toluenesulfonic acid in toluene or benzene. This reaction may be typically conducted under reflux conditions, more generally from about 50° C. and 150° C. The product thus formed may be isolated by basifying an acidic aqueous solution of the salt form of the product and extracting the aqueous solution with a suitable water immiscible solvent. The resulting residue following evaporation can then be further purified if desired.

The 1-alkyl-4-methyl-4-(3-alkoxyphenyl)tetrahydropyridine derivatives may be prepared by a metalloenamine alkylation. This reaction is preferably conducted with n-butyl lithium in tetrahydrofuran (THF) under an inert atmosphere, such as nitrogen or argon. Generally, a slight excess of n-butyl lithium may be added to a stirring solution of the 1-alkyl-4-(3-alkoxyphenyl)-tetrahydropyridine in THF cooled to a temperature in the range of from about −50° C. to about 0° C., more preferably from about −20° C. to −10° C. This mixture may be stirred for approximately 10 to 30 minutes followed by the addition of approximately from 1.0 to 1.5 equivalents of methyl halide to the solution while maintaining the temperature of the reaction mixture below 0° C. After about 5 to 60 minutes, water may be added to the reaction mixture and the organic phase may be collected. The product can be purified according to standard procedures, but the crude product is preferably purified by either distilling it under vacuum or slurrying it in a mixture of hexane:ethyl acetate (65:35, v:v) and silica gel for about two hours. According to the latter procedure, the product may be then isolated by filtration followed by evaporating the filtrate under reduced pressure.

The next step in the process may involve the application of the Mannich reaction of aminomethylation to non-conjugated, endocyclic enamines. This reaction is preferably carried out by combining from about 1.2 to 2.0 equivalents of aqueous formaldehyde and about 1.3 to 2.0 equivalents of a suitable secondary amine in a suitable solvent. While water may be the preferred solvent, other non-nucleophilic solvents, such as acetone and acetonitrile can also be employed in this reaction. The pH of this solution may be adjusted to approximately 3.0 to 4.0 with an acid that provides a non-nucleophilic anion. Examples of such acids include sulfuric acid, the sulfonic acids such as methanesulfonic acid and p-toluenesulfonic acid, phosphoric acid, and tetrafluoroboric acid, with sulfuric acid being preferred. To this solution may be added one equivalent of a 1-alkyl-4-methyl-4-(3-alkoxyphenyl)tetrahydropyridine, typically dissolved in aqueous sulfuric acid, and the pH of the solution may be readjusted with the non-nucleophilic acid or a suitable secondary amine. The pH is preferably maintained in the range of from about 1.0 to 5.0, with a pH of about 3.0 to 3.5 being more preferred during the reaction. The reaction is substantially complete after about 1 to 4 hours, more typically about 2 hours, when conducted at a temperature in the range of from about 50° C. to about 80° C., more preferably about 70° C. The reaction may then be cooled to approximately 30° C., and added to a sodium hydroxide solution. This solution may then be extracted with a water immiscible organic solvent, such as hexane or ethyl acetate, and the organic phase, following thorough washing with water to remove any residual formaldehyde, may be evaporated to dryness under reduced pressure.

The next step of the process may involve the catalytic hydrogenation of the prepared 1-alkyl-4-methyl-4-(3-alkoxyphenyl)-3-tetrahydropyridinemethanamine to the corresponding trans-1-alkyl-3,4-dimethyl-4-(3-alkoxyphenyl)piperidine. This reaction actually occurs in two steps. The first step is the hydrogenolysis reaction wherein the exo C—N bond is reductively cleaved to generate the 3-methyltetrahydropyridine. In the second step, the 2,3-double bond in the tetrahydropyridine ring is reduced to afford the desired piperidine ring.

Reduction of the enamine double bond introduced the crucial relative stereochemistry at the 3 and 4 carbon atoms of the piperidine ring. The reduction generally does not occur with complete stereoselectivity. The catalysts employed in the process may be chosen from among the various palladium and preferably platinum catalysts.

The catalytic hydrogenation step of the process is preferably conducted in an acidic reaction medium. Suitable solvents for use in the process include the alcohols, such as methanol or ethanol, as well as ethyl acetate, tetrahydrofuran, toluene, hexane, and the like.

Proper stereochemical outcome may be dependent on the quantity of catalyst employed. The quantity of catalyst required to produce the desired stereochemical result may be dependent upon the purity of the starting materials in regard to the presence or absence of various catalyst poisons.

The hydrogen pressure in the reaction vessel may not be critical but can be in the range of from about 5 to about 200 psi. Concentration of the starting material by volume is preferably about 20 mL of liquid per gram of starting material, although an increased or decreased concentration of the starting material can also be employed. Under the conditions specified herein, the length of time for the catalytic hydrogenation may not be critical because of the inability for over-reduction of the molecule. While the reaction can continue for up to about 24 hours or longer, it may not be necessary to continue the reduction conditions after the uptake of the theoretical two moles of hydrogen. The product may then be isolated by filtering the reaction mixture for example through infusorial earth, and evaporating the filtrate to dryness under reduced pressure. Further purification of the product thus isolated may not be necessary and preferably, the diastereomeric mixture may be carried directly on to the following reaction.

The alkyl substituent may be removed from the 1-position of the piperidine ring by standard dealkylation procedures. Preferably, a chloroformate derivative, especially the vinyl or phenyl derivatives, may be employed and removed with acid. Next, the prepared alkoxy compound may be dealkylated to the corresponding phenol. This reaction may be generally carried out by reacting the compound in a 48% aqueous hydrobromic acid solution. This reaction may be substantially complete after about 30 minutes to about 24 hours when conducted at a temperature of from about 50° C. to about 150° C., more preferably at the reflux temperature of the reaction mixture. The mixture may then be worked up by cooling the solution, followed by neutralization with base to an approximate pH of 8. This aqueous solution may be extracted with a water immiscible organic solvent. The residue following evaporation of the organic phase may then be used directly in the following step.

The compounds employed as starting materials to the compounds of the invention can also be prepared by brominating the 1-alkyl-4-methyl-4-(3-alkoxyphenyl)-3-tetrahydropyridinemethanamine at the 3-position, lithiating the bromo compound thus prepared, and reacting the lithiated intermediate with a methylhalide, such as methyl bromide to provide the corresponding 1-alkyl-3,4-dimethyl-4-(3-alkoxyphenyl) tetrahydropyridinemethanamine. This compound may then be reduced and converted to the starting material as indicated above.

As noted above, the compounds of the present invention can exist as the individual stereoisomers. Preferably, reaction conditions are adjusted as disclosed in U.S. Pat. No. 4,581,456 or as set forth in Example 1 of U.S. Pat. No. 5,250,542 to be substantially stereoselective and provide a racemic mixture of essentially two enantiomers. These enantiomers may then be resolved. A procedure which may be employed to prepare the resolved starting materials used in the synthesis of these compounds includes treating a racemic mixture of alkyl-3,4-dimethyl-4-(3-alkoxyphenyl)piperidine with either (+)- or (−)-ditoluoyl tartaric acid to provide the resolved intermediate. This compound may then be dealkylated at the 1-position with vinyl chloroformate and finally converted to the desired 4-(3-hydroxyphenyl)piperidine isomer.

Alternatively, the stereoselective syntheses of 3,4-alkyl-substituted-4-(3-hydroxyphenyl)piperidines could be performed by the methods described by Werner, J. A., et al., Journal of Organic Chemistry, 61, 587-597 (1996) and U.S. Pat. No. 5,136,040 using alkoxyphenyllithium (−20° C. to −100° C.) or the corresponding Grignard reagents (40° C. to 60° C.) and 1,3-dialkyl-4-piperidone.

Acylation of the resulting alcohol with ethyl chloloroformate gave the racemic carobante which was efficiently resolved with (+)-di-p-toluoyl-D-tartaric acid (DTTA). Thermal elimination (170-200° C.) of freebase of the chirally pure carbonate gave the desired olefin.

For example, methylation of the olefin with dimethyl sulfate in presence of n-butyl lithium gave the trans-3,4-dimethyl enamine. The reduction of enamine with sodium borohydride followed by purification (+)-DTTA gave the compound with enantiomeric purity >99.5%. Demethylation of the free base with phenyl chloroformate followed by removal of protecting groups resulted in the (3R,4R)-3-(3,4-dimethyl-4-piperidinyl)phenol, a key intermediate for the preparation of compounds of formula I. Alvimopan is manufactured by the process described in Journal of Organic Chemistry, 61, 587-597 (1996) and U.S. Pat. No. 5,136,040.

As will be understood by those skilled in the art, the individual enantiomers of the invention can also be isolated with either (+) or (−) dibenzoyl tartaric acid, as desired, from the corresponding racemic mixture of the compounds of the invention. Preferably, the (+)-trans enantiomer is obtained.

Although the (+)trans-3,4 stereoisomer is preferred, all of the possible stereoisomers of the compounds described herein are within the contemplated scope of the present invention. Racemic mixtures of the stereoisomers as well as the substantially pure stereoisomers are within the scope of the invention. The term “substantially pure,” as used herein, refers to at least about 90 mole percent, more preferably at least about 95 mole percent and most preferably at least about 98 mole percent of the desired stereoisomer is present relative to other possible stereoisomers.

Intermediates can be prepared by reacting a 3,4-alkyl-substituted-4-(3-hydroxyphenyl)piperidine with a compound of the formula LCH₂(CH₂)ₙ₋₁CHR³C(O)E where L is a leaving group such as chlorine, bromine or iodine, E is a carboxylic acid, ester or amide, and R³ and n are as defined hereinabove. Preferably, L may be chlorine and the reaction is carried out in the presence of a base to alkylate the piperidine nitrogen. For example 4-chloro-2-cyclohexylbutanoic acid, ethyl ester can be contacted with (3R,4R)-4-(3-hydroxyphenyl)-3,4-dimethylpiperidine to provide 4-[(3R,4R)-4-(3-hydroxyphenyl) -3,4-dimethyl-1-piperidine]butanoic acid, ethyl ester. Although the ester of the carboxylic acid may be preferred, the free acid itself or an amide of the carboxylic acid may be used.

In alternative synthesis, the substituted piperidine can be contacted with a methylene alkyl ester to alkylate the piperidine nitrogen. For example, 2-methylene-3-phenylproponic acid, ethyl ester can be contacted with a desired piperidine to provide 2-benzyl-3-piperidinepropanoic acid ethyl ester.

Another synthetic route can involve the reaction of a substituted piperidine with a haloalkylnitrile. The nitrile group of the resulting piperidine alkylnitrile can be hydrolyzed to the corresponding carboxylic acid.

With each of the synthetic routes, the resulting ester or carboxylic acid can be reacted with an amine or alcohol to provide modified chemical structures. In the preparation of amides, the piperidine-carboxylic acid or piperidine-carboxylic acid ester may be reacted with an amine in the presence of a coupling agent such as dicyclohexylcarbodiimide, boric acid, borane-trimethylamine, and the like. Esters can be prepared by contacting the piperidine-carboxylic acid with the appropriate alcohol in the presence of a coupling agent such as p-toluenesulfonic acid, boron trifluoride etherate or N,N′-carbonyldiimidazole. Alternatively, the piperidine-carboxylic acid chloride can be prepared using a reagent such as thionyl chloride, phosphorus trichloride, phosphorus pentachloride and the like. This alkanoyl chloride can be reacted with the appropriate amine or alcohol to provide the corresponding amide or ester.

The compounds of formula I are combined with a pharmaceutical acceptable bulking agent selected on the basis of the chosen route of administration and standard pharmaceutical practice as described, for example, in Remington's Pharmaceutical Sciences (Mack Publishing Co., Easton, Pa., 1980), the disclosures of which is hereby incorporated herein by reference, in its entirety.

Compounds of formula I can be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The amount of active compound(s) in such therapeutically useful compositions is preferably such that a suitable dosage will be obtained. Preferred compositions or preparations according to the present invention may be prepared so that a dosage unit form contains from about 0.1 to about 1000 mg of active compound, more preferable from about 1 to 100 mg of the active compound.

Based on the intended use, these preparations may contain a preservative to prevent the growth of microorganisms.

The composition of the invention may be orally administered. For example, the composition of the invention may be enclosed in hard or soft shell gelatin capsules, it may be compressed into tablets (such as fast-dissolve oral tablets, oral disintegrating tablets, including those for buccal administration), or it may be incorporated directly with the food of the diet.

The tablets, troches, pills, capsules and the like for oral administration may also contain one or more of the following provided that they do not interfere with improved solubility and bioavailability of the composition: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

As noted above, the relative proportions of active ingredient and carrier may be determined, for example, by the solubility and chemical nature of the compounds, chosen route of administration, and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached.

The combination products of this invention, such as pharmaceutical compositions comprising opioids in combination with a peripheral p opioid antagonist compound, such as the compounds of formula I, may be in any solid dosage form, such as those described herein, and can also be administered in various ways, as described herein. In a preferred embodiment, the combination products of the invention are formulated together, in a single dosage form (that is, combined together in one solid form, etc.). When the combination products are not formulated together in a single dosage form, the opioid compounds and the peripheral μ opioid antagonist compounds may be administered at the same time or simultaneously (that is, together), or in any order. When not administered at the same time or simultaneously, that is, when administered sequentially, preferably the administration of a peripheral μ opioid antagonist and opioid occurs less than about one hour apart, more preferably less than about 30 minutes apart, even more preferably less than about 15 minutes apart, and still more preferably less than about 5 minutes apart.

Although it is preferable that the peripheral μ opioid antagonists and opioids are administered in the same fashion (that is, for example, both parenterally), if desired, they may each be administered in different fashions (that is, for example, the opioid component of the combination product may be administered orally, and peripheral μ opioid antagonist component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Although the proper dosage of the combination products of this invention will be readily ascertainable by one skilled in the art, once armed with the present disclosure, by way of general guidance, where an opioid compounds is combined with a peripheral μ opioid antagonist, for example, typically a daily dosage may range from about 0.01 to about 100 milligrams of the opioid (and all combinations and subcombinations of ranges therein) and about 0.001 to about 100 milligrams of the peripheral μ opioid antagonist (and all combinations and subcombinations of ranges therein) per kilogram of patient body weight. Preferably, the a daily dosage may be about 0.1 to about 10 milligrams of the opioid and about 0.01 to about 10 milligrams of the peripheral μ opioid antagonist per kilogram of patient body weight. Even more preferably, the daily dosage may be about 1.0 milligrams of the opioid and about 0.1 milligrams of the peripheral μ opioid antagonist per kilogram of patient body weight. With regard to a typical dosage form of this type of combination product, the opioid compounds (e.g., morphine) generally may be present in an amount of about 5 to about 200 milligrams and the peripheral μ opioid antagonists in an amount of about 0.1 to about 12 milligrams.

Pharmaceutical kits useful in, for example, the treatment of the side effects of opioid administration or treatment of pain, which comprise a therapeutically effective amount of an opioid along with a therapeutically effective amount of a peripheral μ opioid antagonist compound, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL™ two-part container (available from Abbott Labs, Chicago, Ill.), as desired. The optional opioid compound and the peripheral μ opioid antagonist compound may be separate, or combined into a single dosage form as described above. Such kits may further include, if desired, one or more of various conventional pharmaceutical kit components, such as for example, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

Compounds for use in the methods and compositions of the present invention, including the compounds of formula I, have been characterized in opioid receptor binding assays showing preferential binding to μ opioid receptors. Studies in isolated tissues (guinea pig ileum and mouse vas deferens) and in other in vitro systems (e.g., GTPγS) have shown that these compounds may act as antagonists with no measurable agonist activity. Studies in animals have demonstrated that the present compounds may reverse constipation in morphine-dependent mice when administered orally or parenterally at very low doses, and do not block the analgesic actions of morphine unless given in hundred-fold or higher doses. Collectively, the data indicate that the compounds described herein may have a very high degree of peripheral selectivity.

### EXAMPLES

The present invention will now be illustrated by reference to the following specific, non-limiting examples. The examples are not intended to limit the scope of the present invention.

### Example 1

### Synthesis of Alvimopan

Alvimopan was prepared in accordance with the following synthetic procedure.

Synthesis of 1-bromo-3-(1-methylethoxy)benzene (Compound 1)

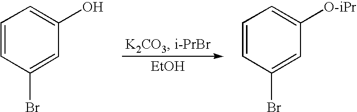

| | | Amount | | Molar |
|---|---|---|---|---|
| Reagent | MW | (kg) | Kilomoles | Ratio |
| 3-Bromophenol | 173.01 | 80.0 | 0.4624 | 1.00 |
| 2-Bromopropane | 123.0 | 85.6 | 0.6959 | 1.51 |
| Potassium carbonate, ground | 138.2 | 96.0 | 0.6946 | 1.50 |
| Ethanol 1X* | 46.07 | 144 | — | — |
| Water | 18.02 | 739 | — | — |
| Hydrochloric acid, 31% | 36.46 | 6.6 | — | — |
| Sodium hydroxide, 50% w/w | 40.0 | 44.4 | — | — |
| Heptanes | 100.2 | 185 | — | — |

*Ethanol 1X was denatured with 0.5% toluene.

A reactor was charged with ground potassium carbonate (96.0 kg) and ethanol 1× (134 kg). The reaction mixture was adjusted to 20 to 25° C.

With agitation, 3-bromophenol (80.0 kg) was charged to the reactor while maintaining the temperature between 20 to 35° C. The transfer equipment was rinsed forward with ethanol 1× (5 kg). The temperature was adjusted to 20 to 25° C. 2-Bromopropane (85.6 kg) was charged to the reactor. The transfer equipment was rinsed forward with ethanol 1× (5 kg). Water (20 L) was charged to the reactor.

The solution in the reactor was heated to 60 to 65° C. and maintained in that range for a minimum of 16 hours. The mixture was cooled to 45 to 50° C. and the mixture was verified for 3-bromophenol. The mixture was warmed to 60 to 65° C. while awaiting the results. The mixture was cooled to 45 to 50° C. once more.

Water (303 L) was charged to the reactor. The reaction mixture was reduced to a concentrate volume of 400 L via atmospheric distillation. The concentrated mixture was cooled to 20 to 25° C.

Heptanes (185 kg) were charged to the reactor and then stirred at a temperature of 20 to 25° C. for a minimum of 20 minutes.

The biphasic solution was separated and the organic layer was washed with a solution of water (45 L) and hydrochloric acid, 31% (6.6 kg). The organic layer was washed with water (56 L) followed by a solution of water (49 L) and sodium hydroxide, 50% (4.4 kg). The organic layer was washed one final time with water (56 L).

The organic solution was dried via azeotropic distillation until no more water was collected. The reaction mixture was then reduced to a concentrate volume of 150 to 170 L via atmospheric distillation and cooled to 20 to 25° C. The solution was packaged for use in the next step. The packaged product (Compound 1) was sampled, tested: HPLC purity not less than 98% a/a and HPLC assay not less than 55% w/w.

Synthesis of cis-(±)-1,3-dimethyl-4-[3-(1-methylethoxy)phenyl]-4-piperidinol (Compound 2)

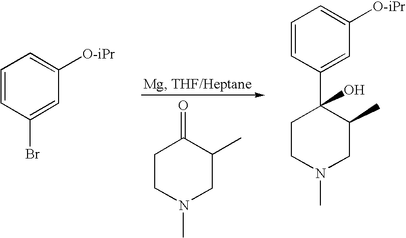

| | | | | Molar |
|---|---|---|---|---|
| Reagent | MW | Amount (kg) | Kilomoles | Ratio |
| Compound 1 | 215.1 | 27.9 | 0.07514* | 1.21 |
| Magnesium turnings | 24.3 | 2.1 | 0.08642 | 1.39 |
| 1,3-Dimethyl-4-piperidone | 127.2 | 7.9 | 0.06211 | 1.00 |
| Tetrahydrofuran | 72.01 | 162 | — | — |
| Ammonium chloride | 53.5 | 6.6 | — | — |
| Water | 18.02 | 56 | — | — |
| Hyflo supercel | — | 4 | — | — |
| Heptanes | 100.2 | 86.5 | — | — |

*Calculated as per assay of reagent

The tetrahydrofuran to be used was sampled for water content prior to use in the lot.

A reactor was charged with tetrahydrofuran (18 kg) and heated to reflux without agitation. The solvent was maintained at reflux for 1 hour and cooled to 30° C. or less. A KF analysis was performed to ensure that the amount of water in the reactor meets the specifications. The THF was drained to waste and the reactor was dried.

Magnesium (2.1 kg) was charged to the reactor, followed by tetrahydrofuran (80 kg). With agitation, the reaction mixture was reduced to a concentrate volume of 40 to 44 L via atmospheric distillation. The concentrate was cooled to 40 to 45° C.

A portable agitation stainless steel tank was charged with tetrahydrofuran (18 kg) and agitated for a minimum of 20 minutes. A KF analysis was performed to ensure that the amount of water in the reactor meets the specifications. The THF was drained to waste.

The tank was charged with 1-bromo-3-(1-methylethoxy)benzene (27.9 kg) and tetrahydrofuran (31 kg). The solution was agitated at room temperature for a minimum of 20 minutes.

A 2.5 kg portion of the mixture in the tank was transferred into the reactor starting at a temperature of 40 to 45° C. With agitation, the mixture was maintained at 40 to 60° C. for a minimum of 30 minutes.

A second 2.5 kg portion of the mixture in the tank was transferred into the reactor starting at a temperature of 40 to 45° C. With agitation, the mixture was maintained at 40 to 60° C. for a minimum of 30 minutes.

A 5 kg portion of the mixture in the tank was transferred into the reactor starting at a temperature of 40 to 45° C. With agitation, the mixture was maintained at 40 to 60° C. for a minimum of 30 minutes.

The tank was charged with 1,3-dimethyl-4-piperidone (7.9 kg) and the transfer equipment was rinsed forward with tetrahydrofuran (5 kg).

A 15 kg portion of the mixture in the tank was transferred into the reactor over a minimum of 1 hour, starting at a temperature of 40 to 45° C. With agitation, the mixture was maintained at 40 to 60° C. for 15 to 30 minutes. The reaction mixture was cooled to 40 to 45° C.

A second 15 kg portion of the mixture in the tank was transferred into the reactor over a minimum of 1 hour, starting at a temperature of 40 to 45° C. With agitation, the mixture was maintained at 40 to 60° C. for 15 to 30 minutes. The reaction mixture was cooled to 40 to 45° C.

A third 15 kg portion of the mixture in the tank was transferred into the reactor over a minimum of 1 hour, starting at a temperature of 40 to 45° C. With agitation, the mixture was maintained at 40 to 60° C. for 15 to 30 minutes. The reaction mixture was cooled to 40 to 45° C.

The remainder of the mixture in the tank was transferred into the reactor over a minimum of 1 hour, starting at a temperature of 40 to 45° C. The transfer equipment was rinsed forward with THF (5 kg). With agitation, the mixture was maintained at 40 to 60° C. for 15 to 30 minutes. The mixture was cooled to 40 to 45° C.

After the reaction was complete, the mixture was cooled to 20 to 25° C.

A second reactor was charged with water (40 L) and ammonium chloride (6.6 kg). With moderate agitation, the mixture was maintained at 20 to 25° C. for a minimum of 20 minutes.

Once the solids have dissolved, Hyflo supercel (4 kg) was charged into the second reactor. The aqueous mixture was cooled to 0 to 5° C.

With agitation, the organic mixture in the first reactor was transferred through to the second reactor. The transfer equipment was rinsed forward with THF (5 kg). The mixture was warmed to 20 to 25° C. and maintained for a minimum of 15 minutes.

The mixture was filtered into the first reactor, rinsed forward with heptanes (2×6 kg), and maintained at 20 to 25° C. for a minimum of 20 minutes.

The biphasic solution was separated and the organic layer was washed with water (16 L). The organic solution was reduced to a concentrate volume of 30 to 34 L via atmospheric distillation and cooled to 45 to 50° C.

Heptanes (54 kg) was charged to the reactor and the solution was reduced to a concentrate volume of 69 to 73 L via atmospheric distillation. The solution was cooled to 30 to 35° C. The reaction mixture was verified for residual tetrahydrofuran and water content. Reaction was seeded with crystals of the product and the mixture was cooled to 0 to 5° C. over a minimum of 1 hour and maintained for a minimum of 3 hours.

The solid product was isolated via filtration, washed with cold heptanes (2×10 kg) and dried. The product was sampled for dryness and packaged. The packaged product (Compound 2) was sampled, tested: HPLC purity not less than 97% a/a and released prior to use in the next step.

Purification of cis-(±)-1,3-dimethyl-4-[3-(1-methylethoxy)phenyl]-4-piperidinol (Compound 2)

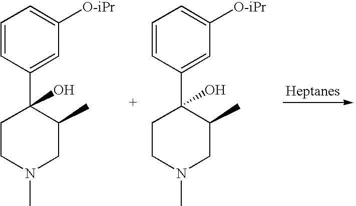

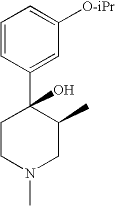

| | | Amount | | Molar |
|---|---|---|---|---|
| Reagent | MW | (kg) | Kilomoles | Ratio |
| Compound 2 | 263.4 | 96.1 | 0.3648 | 1.00 |
| Heptanes | 100.2 | 590 | — | — |

A reactor was charged with compound 2 (96.1 kg) and heptanes (328 L). The mixture was heated to 55 to 60° C. and maintained for a minimum of 1 hour. The mixture was verified to ensure that all of the solids have dissolved.

The solution was cooled to 30 to 35° C. over a minimum of 1 hour and maintained for a minimum of 1 hour. The mixture was verified to ensure that precipitation has occurred. The mixture was cooled to 0 to 5° C. over a minimum of two hours and maintained for a minimum of 4 hours.

The solid purified compound 2 was isolated via filtration, washed with cold heptanes (2×131 kg) and dried. The product was sampled for dryness and packaged. The packaged product was sampled, tested for HPLC purity, not less than 97% a/a and released prior to use in the next step.

Synthesis of carbonic acid, ethyl (3S,4R)-1,3-dimethyl-4-[3-(1-methylethoxy) phenyl]-4-piperidinyl ester compound with (+)-D-2,3-bwas[(4-methylbenzoyl) oxy]butanedioic acid (1:1) (Compound 3)

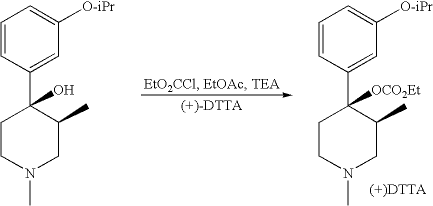

| | | Amount | | Molar |
|---|---|---|---|---|
| Reagent | MW | (kg) | Kilomoles | Ratio |
| Compound 2 | 263.4 | 10.8 | 0.04100 | 1.00 |
| Ethyl chloroformate | 108.52 | 5.6 | 0.05160 | 1.26 |
| Triethylamine, | 101.19 | 0.4 | 0.003953 | 0.10 |
| anhydrous | | | | |
| (+) DTTA | 386.36 | 15.8 | 0.04089 | 1.00 |
| Sodium hydroxide, | 40.0 | 47.6 | — | — |
| 50% w/w | | | | |
| Ethyl acetate | 88.11 | 52 | — | — |
| Ethanol 1X | 46.07 | 285 | — | — |

A reactor was charged with compound 2 (10.8 kg) and ethyl acetate (48 kg). The mixture was maintained at 20 to 25° C. for a minimum of 30 minutes until all of the solids have dissolved. The solution was cooled to 0 to 5° C.

Triethylamine (0.4 kg) was charged to the reactor and the transfer equipment was rinsed forward with ethyl acetate (1 kg).

Ethyl chloroformate (5.6 kg) was charged to the reactor while maintaining a temperature of 0 to 15° C. The transfer equipment was rinsed forward with ethyl acetate (3 kg). The mixture was maintained at 20 to 25° C. for a minimum of 3 hours.

Sodium hydroxide, 50% (7.6 kg) was charged to the reactor while maintaining a temperature of 0 to 38° C. The transfer equipment was rinsed forward with water (17 L). The solution was maintained at 20 to 25° C. for a minimum of 20 minutes and the pH of the solution was checked to ensure it was above 10.

The biphasic solution was separated and the organic layer was washed twice with water (22 L). The organic solution was dried via azeotropic distillation, and then reduced to a concentrate volume of 20 to 24 L via atmospheric distillation. The solution was cooled to 40 to 50° C.

Ethanol 1× (60 kg) was charged to the reactor. The solution was reduced to a concentrate volume of 30 to 34 L via atmospheric distillation and cooled to 55 to 60° C.

A glass-lined reactor was charged with (+)-di-p-toluoyl-D-tartaric acid (15.8 kg) and ethanol 1× (51 kg). With moderate agitation, the temperature was adjusted to 60 to 65° C.

The reaction mixture was transferred into the acid solution while maintaining a temperature of 60 to 70° C. The transfer equipment was rinsed forward with ethanol 1× (17 kg). The solution was maintained at 60 to 65° C. for a period of 1 to 1.5 hours. The suspension was cooled to 50 to 55° C. and maintained for a period of 2 to 2.5 hours. The suspension was cooled to 20 to 25° C. over a minimum of 3 hours and maintained for a minimum of 10 hours.

The solid was isolated by filtration, washed with ethanol 1× (17 kg), dried and packaged. The packaged crude product was sampled and tested for chiral purity of compound 3.

A reactor was charged with the crude product and ethanol 1× (as per calculation). The mixture was adjusted to 60 to 65° C. and maintained for a period of 2 to 2.5 hours. The suspension was cooled to 20 to 25° C. over a minimum of 2 hours. The suspension was cooled to 0 to 5° C. and maintained for a minimum of 3 hours.

The solid compound 3 was isolated via filtration, washed with cold ethanol 1× (17 kg), dried and packaged. The packaged product was sampled, tested, HPLC purity not less than 99.0% a/a; Chiral HPLC, not less than 99.5% and released prior to use in the next step.

Synthesis of (3R,4R)-3-(3,4-dimethyl-4-piperidinyl)phenol (Compound 4)

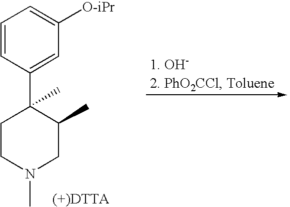

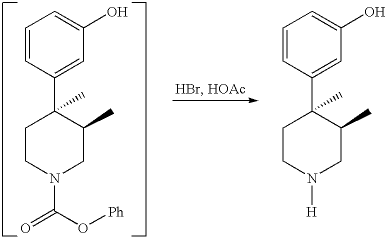

| | | Amount | | Molar |
|---|---|---|---|---|
| Reagent | MW | (kg) | Kilomoles | Ratio |
| Compound 3 | 647.8 | 18.3 | 0.02825 | 1.00 |
| Toluene | 92.14 | 50 | — | — |
| Water | 18.02 | 434 | — | — |
| Sodium | | | | |
| hydroxide, 50% | 40.0 | 110.7 | — | — |
| w/w | | | | |
| Phenyl | 156.57 | 5.3 | 0.03385 | 1.20 |
| chioroformate | | | | |
| Hydrochloric | 36.46 | 2.8 | — | — |
| acid, 31% | | | | |
| Acetic acid, | 60.05 | 17.6 | 0.2931 | 10.38 |
| glacial | | | | |
| Hydrobromic | 80.92 | 19 | 0.1127 | 4.00 |
| acid | | | | |
| t-Butyl methyl | 88.15 | 56 | — | — |
| ether | | | | |
| Methanol | 32.04 | 8.7 | — | — |

A reactor was charged with compound 3 (18.3 kg), toluene (48 kg), and water (32 L). The mixture was adjusted to 20 to 25° C.

Sodium hydroxide, 50% (9.2 kg) was charged to the reactor while maintaining a temperature of 20 to 30° C. The transfer equipment was rinsed forward with water (4 L). With agitation, the mixture was cooled to 20 to 25° C. and maintained for 1 hour. The pH of the aqueous layer was checked to ensure that it was above 12.

The biphasic solution was separated and the organic layer was washed with a solution of water (14 L) and sodium hydroxide, 50% (0.7 kg). The organic layer was washed twice with water (15 L) and dried via azeotropic distillation. The solution was cooled to 80 to 85° C.

Phenyl chloroformate (5.3 kg) was charged to the reactor over a minimum of 1.5 hours while maintaining a temperature of 80 to 85° C. The transfer equipment was rinsed forward with toluene (2 kg). The solution was heated to reflux and maintained for a minimum of 3 hours, then cooled to 50 to 55° C. The mixture was maintained at reflux while awaiting the results.

The mixture was cooled to 20 to 25° C. and water (14 L) was charged to the reactor. Sodium hydroxide, 50% (2.3 kg) was charged to the reactor over a minimum of 1 hour while maintaining a temperature of 20 to 30° C. The transfer equipment was rinsed forward with water (4 L). The solution was maintained at 20 to 25° C. for a minimum of 1 hour.

The biphasic solution was separated and the organic layer was washed with a solution of water (15 L) and hydrochloric acid, 31% (1.9 kg). The organic solution was reduced to a concentrate volume of 23 to 26 L via atmospheric distillation and cooled to 65 to 70° C.

Water (7 L) and acetic acid (13.6 kg) were charged to the reactor. The transfer equipment was rinsed forward with water (2 L). The solution was reduced to a concentrate volume of 26 to 29 L via atmospheric distillation and cooled to 50 to 60° C.

Hydrobromic acid (19 kg) was charged to the reactor, followed by acetic acid (4 kg). The solution was heated to reflux and maintained for a minimum of 18 hours. The solution was cooled to 55 to 60° C. The solution was cooled to 10 to 15° C.

Sodium hydroxide, 50% (6 kg) was charged to the reactor over a minimum of 1 hour while maintaining a temperature of 10 to 30° C. The transfer equipment was rinsed forward with water (5 L). The temperature was adjusted to 20 to 25° C. and the pH was checked to ensure it was less than 1.7.

To the reactor, t-butyl methyl ether (16 kg) was charged while maintaining a temperature of 20 to 25° C. Water (27 L) was charged to the reactor and the solution was maintained at 20 to 25° C. for a minimum of 30 minutes.

The biphasic solution was separated and the aqueous solution was transferred to a reactor. The organic solution was transferred to a 200 L glass receiver. The aqueous solution was washed twice with t-butyl methyl ether (16 kg).

The organic layers were transferred from the glass receiver to a reactor. Water (5 L) was charged to the reactor, followed by hydrochloric acid, 31% (0.9 kg) while maintaining a temperature of 20 to 25° C. The transfer equipment was rinsed forward with water (2 L). The biphasic solution was maintained at 20 to 25° C. for a minimum of 20 minutes.

The biphasic solution was separated and the aqueous solution was washed twice with t-butyl methyl ether (4 kg).

The acidic solution from the new PE drum was transferred to the 200 L reactor. The transfer equipment was rinsed forward with water (2 L).

Methanol (8.7 kg) was charged to the reactor over a minimum of 30 minutes while maintaining a temperature of 20 to 25° C.

A portable agitation stainless steel tank was charged with water (41 L) and sodium hydroxide, 50% (12.5 kg). The transfer equipment was rinsed forward with water (4 L). The solution was transferred to the reactor to achieve a pH of 10.0 to 10.5 while maintaining a temperature of 20 to 35° C.

The suspension was cooled to 0 to 5° C. and maintained for a minimum of 4 hours.

The compound 4 was isolated via filtration, washed with cold water (2×9 L), dried, and packaged. The packaged product was sampled, tested: HPLC Purity, not less than 98.5% a/a; Chiral Purity, not less than 99.0% and HPLC Assay, not less than 95% w/w and released prior to use in the next step.

Synthesis of methyl (αS,3R,4R)-4-(3-hydroxyphenyl)-3,4-dimethyl-α-(phenylmethyl)-1-piperidinepropanoate hydrochloride (Compound 6)

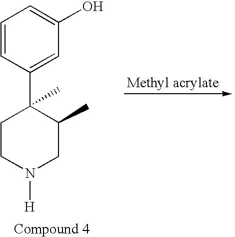

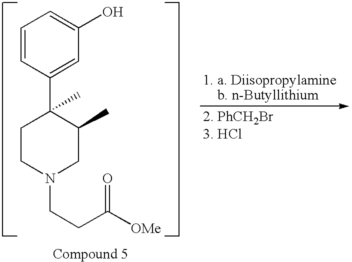

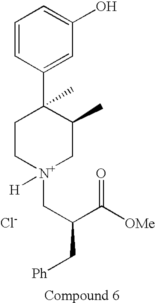

| | | Amount | | Molar |
|---|---|---|---|---|
| Reagent | MW | (kg) | Kilomoles | Ratio |
| Compound 4 | 205.3 | 19.2 | 0.09352 | 1.00 |
| Methyl acrylate | 86.09 | 8.5 | 0.09875 | 1.05 |
| Tetrahydrofuran | 72.11 | 692 | — | — |
| n-Butyllithium | 64.06 | 87.4 | 0.2056 | 2.20 |
| Diisopropylamine | 101.19 | 21.8 | 0.2154 | 2.30 |
| Benzyl bromide | 171.04 | 32.0 | 0.1871 | 2.00 |
| Heptanes | 100.21 | 209 | — | — |
| Methanol | 32.04 | 659 | — | — |
| Hydrochloric | 36.46 | 36.2 | 0.3078 | 3.29 |
| acid, 31% | | | | |
| Sodium | | | | |
| hydroxide, 50% | 40.0 | 4.9 | 0.06125 | 0.65 |
| w/w | | | | |
| Hydrogen | 36.46 | 14.4 | 0.3950 | 4.23 |
| chloride gas | | | | |
| Hyflo supercel | — | 1.9 | — | — |
| Water | 18.02 | 566 | — | — |

A reactor was charged with compound 4(19.2 kg) and tetrahydrofuran (222 kg). The mixture was heated to 40 to 45° C. with 50% agitation.

Methyl acrylate (8.5 kg) was charged to the reactor over a minimum of 30 minutes while maintaining a temperature of 40 to 45° C. The transfer equipment was rinsed forward with THF (17 kg). The reaction mixture was maintained at 40 to 45° C. for a period of 18 to 19 hours. The reaction mixture was cooled to 20 to 25° C.

A portable agitation stainless steel tank was charged with hyflo supercel (1.9 kg) and heptanes (13 kg). The mixture was agitated for a minimum of five minutes. The mixture was transferred to the reactor and rinsed forward with heptanes (5 kg). The mixture was maintained at 20 to 25° C. for a minimum of 20 minutes.

The mixture was filtered into a reactor for clarification, rinsed forward with heptanes (26 kg) and cooled to −5 to 0° C. The solution was reduced to a concentrate volume of 29 to 48 L via vacuum distillation to give a solution of compound 5.

Heptanes (26 kg) was charged to the reactor at 30° C. or less. The solution was cooled to −5 to 0° C. and reduced to a concentrate volume of 29 to 48 L via vacuum distillation.

Tetrahydrofuran (333 kg) was charged to the reactor, followed by diisopropylamine (21.8 kg). The transfer equipment was rinsed forward with tetrahydrofuran (12 kg). The solution was cooled to −15 to −10° C.

The reactor was charged with n-butyllithium in hexanes (87.4 kg) over a minimum of 1 hour while maintaining a temperature of −15 to −5° C. The transfer equipment was rinsed forward with THF (2×5 kg). The solution was maintained at −10 to −5° C. for a period of 1 to 3 hours, then cooled to −25 to −20° C.

The acrylate solution in the reactor was transferred to this reactor while maintaining a temperature of −25 to −15° C. The transfer equipment was rinsed forward with THF (8 kg). The suspension was maintained at −25 to −20° C. for a period of 30 to 60 minutes.

Benzyl bromide (32.0 kg) was charged to the reactor over a minimum of 2 hours while maintaining a temperature of −25 to −20° C. The transfer equipment was rinsed forward with THF (8 kg). The mixture was maintained at −25 to −20° C. for a minimum of 16 hours.

A portable storage tank was charged with water (61 L) and hydrochloric acid, 31% (18.1 kg), and then agitated for a minimum of two minutes to form a solution. A second portable storage tank was charged with water (61 L) and hydrochloric acid, 31% (18.1 kg), and then agitated for a minimum of two minutes to form a solution. Both acid solutions were transferred to the reactor over a minimum of two hours while maintaining a temperature of −25 to −15° C. The solution was warmed to 20 to 25° C. over a minimum of three hours.

A portable storage tank was charged with water (29 L) and sodium hydroxide, 50% (4.9 kg). The transfer equipment was rinsed forward with water (15 L) and the mixture was agitated for a minimum of two minutes to form a solution.

The basic solution (29 kg) was transferred to the reactor while maintaining a temperature of 20 to 25° C. until a pH of 9.0 to 9.5 was obtained. The biphasic solution was separated and the aqueous solution was transferred to the 600 L reactor.

The aqueous solution was washed with heptanes (26 kg). The resulting organic solution was transferred to the 1500 L reactor and the transfer equipment was rinsed forward with heptanes (17 kg). The solution was cooled to −30 to −20° C.

A reactor was charged with methanol (113 kg) and cooled to −30 to −20° C. Hydrogen chloride gas (14.4 kg) was charged to the reactor while maintaining a temperature of −30 to −10° C.

The acid solution was charged to above reactor while maintaining a temperature of −30 to −5° C. The transfer equipment was rinsed forward with methanol (19 kg). The solution temperature was adjusted to −10 to −5° C. The solution was reduced to a concentrate volume of 168 to 216 L via vacuum distillation.

The solution was transferred to the 600 L reactor and rinsed forward with methanol (48 kg). The solution was cooled to −10 to −5° C. and reduced to a concentrate volume of 48 to 68 L via vacuum distillation.

Methanol (77 kg) was charged to the 1500 L reactor and rinsed into the reactor. The solution was then cooled to −10 to −5° C. and reduced to a concentrate volume of 48 to 68 L via vacuum distillation.

Methanol (106 kg) was charged to the reactor at a temperature of 30° C. or less, and then heated to 40 to 45° C. The solution was maintained at 40 to 45° C. for a period of 1 to 2 hours. The solution was cooled to 20 to 25° C. over a minimum of 3 hours and maintained in the range for a minimum of 1 hour. The solution was cooled to 2 to 7° C. over a minimum of 1 hour and maintained in the range for a minimum of 1 hour.

The crude product, compound 6, was isolated by filtration, washed with cold methanol (2×15 kg), and tested for purity. The filtrate was kept for further processing.

A reactor was charged with the wet filter cake and methanol (60 kg). The mixture was heated to reflux and maintained at reflux for a period of 1 to 2 hours. The solution was cooled to 2 to 7° C. over a minimum of 4 hours and maintained in the range for a minimum of 1 hour.

The crude product was isolated by filtration, washed with cold methanol (2×15 kg), and tested for purity. The filtrate was kept for further processing.

The reactor was charged with the wet filter cake and methanol (60 kg). The mixture was heated to reflux and maintained at reflux for a minimum of 1 hour. The solution was cooled to 2 to 7° C. over a minimum of 4 hours and maintained in the range for a minimum of 1 hour.

The crude product was isolated by filtration, washed with cold methanol (2×15 kg), and tested for purity and chiral HPLC

The reactor was charged with the wet filter cake and methanol (60 kg). The mixture was heated to reflux and maintained at reflux for a minimum of 1 hour. The solution was cooled to 2 to 7° C. over a minimum of 4 hours and maintained in the range for a minimum of 1 hour.

The product compound 6 was isolated by filtration, washed with cold methanol (2×15 kg), sampled for HPLC purity, Chiral HPLC, and isomers and packaged. The packaged product was sampled, tested: HPLC purity; >99.0% a/a and Chiral HPLC, 3.0% and released before use in the next step.

Synthesis of (αS,3R,4R)-4-(3-hydroxyphenyl)-3,4-dimethyl-α-(phenylmethyl)-1-piperidinepropanoic acid monohydrate (Compound 7)

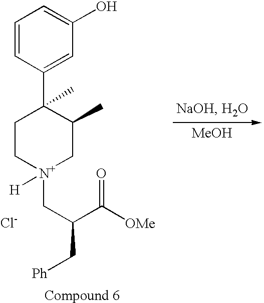

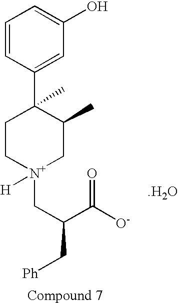

| | | Amount | | Molar |
|---|---|---|---|---|
| Reagent | MW | (kg) | Kilomoles | Ratio |
| Compound 6 | 417.97 | 9.9 | 0.02369 | 1.00 |
| Methanol | 32.04 | 107 | — | — |
| Hydrochloric | 36.46 | 9.4 | 0.07992 | 3.37 |
| acid, 31% | | | | |
| Sodium | | | | |
| hydroxide, 50% | 40.0 | 7.9 | 0.09875 | 4.16 |
| w/w | | | | |
| Water | 18.02 | ˜244 | — | — |

A reactor was charged with compound 6 (9.9 kg) and water (74 L). The mixture was adjusted to 20 to 25° C.

Sodium hydroxide, 50% (7.9 kg) was charged to the reactor over a minimum of 10 minutes. The transfer equipment was rinsed forward with water (10 L). The pH of the mixture was checked to ensure it was above 12.

The solution was maintained and agitated at a temperature of 20 to 25° C. for a minimum of 4 hours. The reaction mixture was then filtered into a reactor for clarification. The product was rinsed forward with water (8 L).

Methanol (84 kg) was charged to the reactor and adjusted to 20 to 25° C.

Hydrochloric acid, 31% (6.9 kg) was charged to the reactor in portions until a pH of 9.0 to 10.0 was reached.

A new PE drum was charged with water (7.6 L) and hydrochloric acid, 31% (2.5 kg). The transfer equipment was rinsed forward with water (4.0 L) and the solution was agitated for a minimum of two minutes to mix.

A beaker was charged with methanol (0.4 kg), water (0.5 L), and Compound 7(100 g). The mixture was charged to the reactor and rinsed forward with a solution of water (0.3 L) and methanol (0.2 kg) to seed the reaction mixture.

The pH of the reaction mixture was adjusted with the prepared acidic solution (10.4 kg) until a pH of 5.8 to 6.2 was obtained. The mixture was maintained at 20 to 25° C. for a minimum of 1 hour and verified to ensure crystallization has occurred. The suspension was cooled to 0 to 5° C. and reduced to a concentrate volume of 107 to 124 L via vacuum distillation. The suspension was adjusted to 20 to 25° C. and the pH was checked to ensure it was between 5.8 and 6.2.

The suspension was cooled to 2 to 7° C. and agitated for a minimum of 4 hours.

The product was isolated by filtration, washed with cold water (2×30 L), dried, sampled for water content and packaged. The packaged product was sampled, tested: HPLC purity, 98.% a/a, Chiral HPLC, 98%, and HPLC assay, 98.0% w/w and released prior to use in the next step.

Synthesis of [[2(S)-[[4(R)-(3-hydroxyphenyl)-3(R),4-dimethyl-1-piperidinyl]methyl]-1-oxo-3-phenylpropyl]amino]acetic acid dihydrate (Alvimopan)

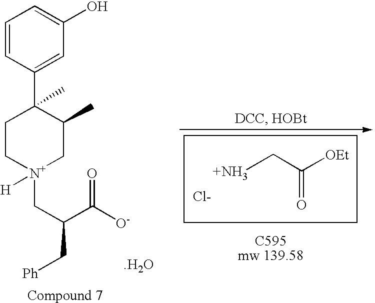

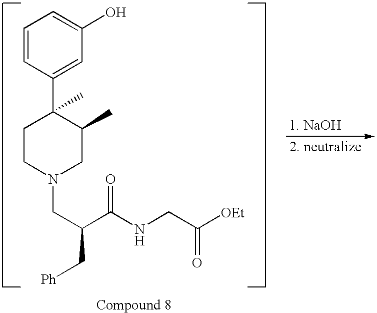

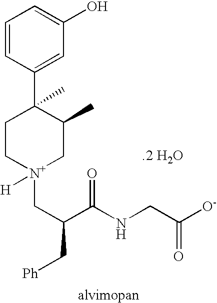

| | | Amount | | Molar |
|---|---|---|---|---|
| Reagent | MW | (kg) | Kilomoles | Ratio |
| Compound 7 | 385.5 | 7.9 | 2.02049 | 1.00 |
| Glycine ethyl ester | 139.58 | 3.1 | 0.02254 | 1.10 |
| hydrochloride | | | | |
| 1-Hydroxybenzotriazole | 135.13 | 3.5 | 0.02562 | 1.25 |
| hydrate | | | | |
| Triethylamine | 101.2 | 2.3 | 0.02254 | 1.10 |
| 1,3- | 206.33 | 4.7 | 0.02254 | 1.10 |
| Dicyclohexylcarbodiimide | | | | |
| Tetrahydrofuran | 72.11 | 156 | — | — |
| Ethyl acetate | 88.11 | 858 | — | — |
| Soda ash (Sodium | 105.99 | 4.8 | — | — |
| carbonate) | | | | |
| Sodium bicarbonate | 84.00 | 3.1 | — | — |
| Brine | — | 112 | — | — |
| Ethanol 1X | 46.07 | 743 | — | — |
| Purifiedwater | 18.02 | 1196 | — | — |
| Sodium hydroxide, 50% | 40.0 | 16.8 | — | — |
| w/w | | | | |
| Hydrochloric acid, 31% | 36.46 | 30.0 | — | — |

A portable agitation stainless steel tank (PAST) was charged with tetrahydrofuran (15 kg) and 1,3-dicyclohexylcarbodiimide (4.7 kg). The transfer equipment was rinsed forward with THF (16 kg).

A reactor was charged with compound 7 (7.9 kg), glycine ethyl ester hydrochloride (3.1 kg), 1-hydroxybenzotriazole hydrate (3.5 kg), tetrahydrofuran (99 kg) and purified water (3.3 kg). With 60% agitation, the mixture was adjusted to 20 to 25° C.

Triethylamine (2.3 kg) was charged to the reactor. The transfer equipment was rinsed forward with tetrahydrofuran (3 kg). The solution was maintained at 20 to 25° C. for a period of 20 to 60 minutes.

The 1,3-dicyclohexylcarbodiimide solution was transferred to the reactor while maintaining a temperature of 20 to 25° C. The transfer equipment was rinsed forward with tetrahydrofuran (23 kg).

The reaction mixture was maintained at 20 to 25° C. for a period of 36 to 38 hours with 100% agitation.

The reaction mixture was cooled to 0 to 5° C. The mixture was maintained in range for a period of 1 to 2 hours then filtered into another reactor. The reaction mixture was rinsed forward with ethyl acetate (20 kg). The mixture was cooled to 0 to 5° C. and reduced to a concentrate volume of 140 to 149 L via vacuum distillation.

Ethyl acetate (731 kg) was charged to the reactor and cooled to 0 to 5° C. The solution was reduced to a concentrate volume of 140 to 149 L via vacuum distillation. The mixture was verified for residual tetrahydrofuran.

A portable agitation stainless steel tank was charged with purified water (94 kg), soda ash (4.8 kg) and sodium bicarbonate (3.1 kg). The mixture was agitated for a minimum of two minutes until the solids dissolved.

The basic solution was charged to the reactor and the temperature was adjusted to 20 to 25° C. The agitation was maintained at 60% for a period of 20 to 60 minutes. The pH of the solution was checked to ensure it was between 9.5 and 10, and adjusted as necessary. The biphasic solution was separated and the organic solution was washed with brine (112 kg).

The reactor was charged with ethyl acetate (87 kg) and cooled to 0 to 5° C. The solution was reduced to a concentrate volume of 140 to 149 L via vacuum distillation, and cooled to −25 to −20° C. The temperature was maintained for a period of 10 to 11 hours.

The suspension was filtered into a reactor, rinsed forward with ethyl acetate (20 kg) and warmed to 0 to 5° C. The filtrate was reduced to a concentrate volume of 39 to 51 L via vacuum distillation.

Ethanol 1× (199 kg) was charged to the reactor and cooled to 0 to 5° C. The solution was reduced to a concentrate volume of 136 to 161 L via vacuum distillation. The reactor was charged with ethanol 1× (93 kg) and the mixture was verified for residual ethyl acetate.

A portable storage tank was charged with purified water (83 kg) and sodium hydroxide, 50% (5.6 kg). The transfer equipment was rinsed forward with purified water (19 kg). The mixture was agitated for a minimum of two minutes to form a solution. The basic solution was transferred to the reactor and maintained at 20 to 25° C. for a period of 1.5 to 3.5 hours. The suspension was filtered into a reactor and adjusted to 20 to 25° C. The 600 L reactor was rinsed forward with purified water (13 kg).

A portable storage tank was charged with purified water (15 kg) and hydrochloric acid, 31% (11.2 kg). The transfer equipment was rinsed forward with purified water (5 kg). The mixture was agitated for a minimum of two minutes to form a solution. The acidic solution was charged to the reactor in portions until a pH of 5.8 to 6.2 was achieved.

The crude product was isolated by filtration, washed with purified water (2×26 kg), washed with ethanol 1× (13 kg), dried and packaged.

The crude product was charged to a reactor with purified water (as per calculation).

A new PE pail was charged with purified water (1.9 kg) and sodium hydroxide, 50% (5.3 kg). The transfer equipment was rinsed forward with purified water (1.0 kg). The mixture was agitated for a minimum of two minutes to form a solution.

The reaction mixture was adjusted to a minimum pH of 13 using the basic solution (7.5 kg). The mixture was maintained at 20 to 25° C. for a period of 20 to 60 minutes.

The mixture was filtered for clarification into a reactor. The reactor was rinsed forward with purified water (10 kg) and was charged with ethanol 1× (as per calculation).

A portable storage tank was charged with purified water (14 kg) and hydrochloric acid, 31% (9.6 kg). The transfer equipment was rinsed forward with purified water (4 kg). The mixture was agitated for a minimum of two minutes to form a solution. The acidic solution was charged to the reactor in portions until a pH of 4.0 to 4.5 was obtained.

A new PE pail was charged with purified water (1.9 kg) and sodium hydroxide, 50% (0.3 kg). The transfer equipment was rinsed forward with purified water (1.0 kg). The mixture was agitated for a minimum of two minutes to form a solution. The basic solution was charged to the reactor in portions until a pH of 5.8 to 6.2 was obtained.

The mixture was verified for the presence of solids and the suspension was maintained at 20 to 25° C. for a minimum of 12 hours.

The product was isolated by filtration, washed first with purified water (as per calculation), next with ethanol 1× (as per calculation) and washed again with purified water (as per calculation). The filter cake was dried and packaged.

The crude product was charged to a reactor with purified water (as per calculation).

A new PE pail was charged with purified water (1.9 kg) and sodium hydroxide, 50% (5.3 kg). The transfer equipment was rinsed forward with purified water (1.0 kg). The mixture was agitated for a minimum of two minutes to form a solution. The basic solution was charged to the reactor in portions until a minimum pH of 13 was obtained.

The mixture was agitated at 20 to 25° C. for a period of 20 to 60 minutes. The mixture was filtered for clarification into another reactor. The reactor was rinsed forward with purified water (10 kg). The reactor was charged with ethanol 1× (as per calculation).

A portable storage tank was charged with purified water (13.5 kg) and hydrochloric acid, 31% (9.2 kg). The transfer equipment was rinsed forward with purified water (3.9 kg). The mixture was agitated for a minimum of two minutes to form a solution. The acidic solution was charged to the reactor in portions until a pH of 4.0 to 4.5 was obtained.

A new polyethylene pail was charged with purified water (1.9 kg) and sodium hydroxide, 50% (0.3 kg). The transfer equipment was rinsed forward with purified water (1.0 kg). The mixture was agitated for a minimum of two minutes to form a solution. The basic solution was charged to the reactor in portions until a pH of 5.8 to 6.2 was obtained.

The mixture was verified for the presence of solids and the suspension was maintained at 20 to 25° C. for a minimum of 12 hours.

The product was isolated by filtration, washed first with purified water (as per calculation), next with ethanol 1× (as per calculation) and washed again with purified water (as per calculation). The filter cake was sampled for chloride, dried and packaged.

The dryer was charged with the over-dried product and purified water (2.0 kg), flushed with nitrogen and left at room temperature until the specified hydration level was achieved.

The hydrated product was then packaged and charged to a 50 L product blender. The product was blended for a period of twenty to thirty minutes and sampled for dryness. The product was blended for a further twenty to thirty minutes and resampled.

The alvimopan was then packaged, sampled, tested: HPLC purity, not less than 99.2% w/w; Chiral HPLC, not less than 99.0%; HPLC assay, 98.0 to 102.0% w/w and residual solvents, not more than 1.2% w/w total and released.

### Example 2

### Particle Size Distribution

A mixture of alvimopan and mannitol (50% by weight alvimopan and 50% by weight mannitol) was prepared and micronized in an air attrition mill. A comparative mixture of alvimopan and corn starch or colloidal silicon dioxide (50% by weight alvimopan and 50% by weight corn starch or colloidal silicon dioxide) was also prepared and micronized. Particle size distribution of each mixture before and after micronization was determined by laser diffraction and is shown in the following table:

| | Particle Size (μ) | | | Particle Size (μ) | | |
|---|---|---|---|---|---|---|
| | Before Micronization | | | After Micronization | | |
| | ≦10% of | ≦50% of | ≦90% of | ≦10% of | ≦50% of | ≦90% of |
| | particles | particles | particles | particles | particles | particles |
| Mixture | by weight | by weight | by weight | by weight | by weight | by weight |
| alvimopan and | 4.85 | 109.77 | 325.97 | 3.08 | 7.87 | 16.10 |
| mannitol | | | | | | |
| alvimopan and | 30.84 | 217.89 | 488.26 | 2.83 | 9.10 | 190.39 |
| corn starch | | | | | | |
| (comparative) | | | | | | |
| alvimopan and | 22.4 | 101.7 | 293.6 | 14.1 | 58.7 | 149.4 |
| colloidal | | | | | | |
| silicon dioxide | | | | | | |
| (comparative) | | | | | | |

As can be seen, there is a much larger particle size distribution for the comparative mixture of the alvimopan and corn starch than for the inventive mixture of the alvimopan and mannitol.

### Example 3

### Dissolution Studies

A mixture of alvimopan and mannitol was prepared and micronized in an air attrition mill, mixed with microcrystalline cellulose and filled into capsules. The composition of the capsule was as follows: alvimopan/mannitol micronized blend 12 mg and microcrystalline cellulose 188 mg. A comparative mixture of 6 mg alvimopan and 294 mg molten polyethylene glycol (PEG) was also prepared and filled into capsules. The dissolution rate was determined (50 rpm, 0.1 HCl 900 ml) for both types of capsules and the results are shown in the following table:

% Alvimopan Released

| Time | Micronized composition | Comparative PEG |
|---|---|---|
| (minutes) | of invention | Formulation |
| 0 | 0 | 0 |
| 10 | 95 | 49 |
| 15 | 98 | 79 |
| 20 | 100 | 91 |
| 30 | 101 | 94 |

As may be seen from the results, there was a significant improvement in the dissolution rate of the conventional formulation of the compound of formula I with the composition and method of the invention.

When ranges are used herein for physical properties, such as molecular weight, or chemical properties, such as chemical formulae, all combinations and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method, comprising the steps of:
a. providing a composition, comprising:
(i) at least one compound of formula I or a pharmaceutically acceptable salt or stable polymorph thereof: 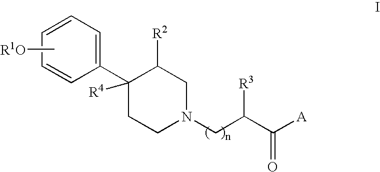
wherein:
R¹ is hydrogen or alkyl;
R²is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, aralkyl, or alkylene substituted B or, together with the nitrogen atom to which they are attached, R⁶ and R⁷ form a heterocyclic ring;
B is 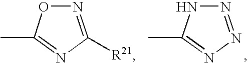
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 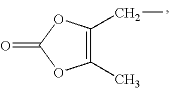
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹is hydrogen or alkyl; and
n is 0 to 4; and
(ii) a pharmaceutically-acceptable excipient selected from the group consisting of mannitol, dextrose, fructose, lactose, sucrose, dextrate, maltodextrin, and mixtures thereof; and
b. micronizing said composition.

2. A method according to claim 1,
wherein the weight ratio of said compound of formula I or a pharmaceutically acceptable salt or stable polymorph thereof to said excipient is about 10:1 to about 1:10.

3. A method according to claim 1,
wherein the weight ratio of said compound of formula I or a pharmaceutically acceptable salt or stable polymorph thereof to said excipient is about 5:1 to about 1:5.

4. A method according to claim 1,
wherein the weight ratio of said compound of formula I or a pharmaceutically acceptable salt or stable polymorph thereof to said excipient is about 2:1 to about 1:2.

5. A method according to claim 1,
wherein the weight ratio of said compound of formula I or a pharmaceutically acceptable salt or stable polymorph thereof to said excipient is about 1:1.

6. A method according to claim 1,
wherein said composition after said micronizing step has an average particle size range of about 5 microns to about 20 microns.

7. A method according to claim 1,
wherein said compound of formula I or a pharmaceutically acceptable salt or stable polymorph thereof has an average particle size range of about 5 microns to about 20 microns.

8. A method according to claim 1,
wherein said excipient has an average particle size range of about 5 microns to about 20 microns.

9. A method according to claim 1,
wherein the average particle size of said compound of formula I or a pharmaceutically acceptable salt or stable polymorph thereof differs from the average particle size of said pharmaceutically acceptable excipient by no more than about 200%.

10. A method according to claim 1,
wherein the average particle size of said compound of formula I or a pharmaceutically acceptable salt or stable polymorph thereof differs from the average particle size of said excipient by no more than about 100%.

11. A method according to claim 1,
wherein the average particle size of said compound of formula I or a pharmaceutically acceptable salt or stable polymorph thereof differs from the average particle size of said excipient by no more than about 50%.

12. A method according to claim 1,
wherein the average particle size of said compound of formula I or a pharmaceutically acceptable salt or stable polymorph thereof differs from the average particle size of said excipient by no more than about 25%.

13. A method according to claim 1,
wherein the compound of formula I is a trans 3,4-isomer.

14. A method according to claim 1,
wherein:
R¹ is hydrogen;
R² is alkyl;
n is 1 or 2;
R³ is benzyl, phenyl, cyclohexyl, or cyclohexylmethyl; and
R⁴ is alkyl.

15. A method according to claim 1,
wherein:
A is OR⁵; and
R⁵ is hydrogen or alkyl.

16. A method according to claim 1,
wherein:
A is NR⁶R⁷;
R⁶ is hydrogen;
R⁷ is alkylene substituted B; and
B is C(O)W.

17. A method according to claim 1,
wherein:
R⁷ is (CH₂)_{q}—B;
q is about 1 to about 3;
W is OR¹⁰; and
R¹⁰ is hydrogen, alkyl, phenyl-substituted alkyl, cycloalkyl or cycloalkyl-substituted alkyl.

18. A method according to claim 1,
wherein:
W is NR¹¹R¹²
R¹¹ is hydrogen or alkyl; and
R¹² is hydrogen, alkyl or alkylene substituted C(═O)Y.

19. A method according to claim 1,
wherein:
R¹² is (CH₂)ₘC(O)Y;
m is 1 to 3;
Y is OR¹⁸ or NR¹⁹R²⁰; and
R¹⁸, R¹⁹, and R²⁰ are independently hydrogen or alkyl.

20. A method according to claim 1,
wherein:
W is OE;
E is CH₂C(═O)D;
D is OR¹⁵ or NR¹⁶ R¹⁷;
R¹⁵ is hydrogen or alkyl;
R¹⁶ is methyl or benzyl; and
R¹⁷ is hydrogen.

21. A method according to claim 1,
wherein:
W is OE;
E is R^{13OC(═O)R}¹⁴;
R¹³ is —CH(CH₃)— or —CH(CH₂CH₃)—; and
R¹⁴ is alkyl.

22. A method according to claim 1,
wherein the configuration at positions 3 and 4 of the piperidine ring is each R.

23. A method according to claim 1,
wherein said compound of formula I is selected from the group consisting of:
Q-CH₂CH(CH₂(C₆H₅))C(O)OH,
Q-CH₂CH₂CH(C₆H₅)C(O)NHCH₂C(O)OCH₂CH₂,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NHCH₂C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OCH₂CH₃,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NHCH₂C(O)OCH₂CH(CH₃)₂,
Z-NH(CH₂)₂C(O)OCH₂(C₆H₅),
Z-NH(CH₂)₂C(O)OH,
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)O(CH₂)₄CH₃,
Z-NHCH₂C(O)OCH₂C(O)NHCH₃,
Z-NHCH₂C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
wherein:
Q represents 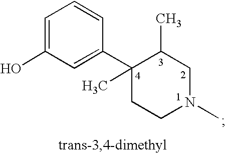
G represents 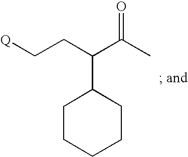
Z represents

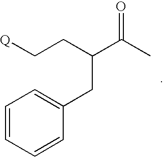

24. A method according to claim 23,
wherein said compound of formula I is selected from the group consisting of:
(3R,4R,S)—Z-NHCH₂C(O)OCH₂CH(CH₃)₂,
(+)—Z-NHCH₂C(O)OH,
(−)—Z-NHCH₂C(O)OH,
(3R,4R,R)—Z-NHCH₂C(O)—OCH₂CH(CH₃)₂,
(3S,4S,S)—Z-NHCH₂C(O)OCH₂CH(CH₃)₂,
(3S,4S,R)—Z-NHCH₂C(O)OCH₂CH(CH₃)₂,
(3R,4R)—Z-NHCH₂C(O)NHCH₂(C₆H₅), and
(3R,4R)-G-NH(CH₂)₃C(O)OH.

25. A method according to claim 23,
wherein said compound of formula I is selected from the group consisting of (+)—Z-NHCH₂C(O)OH and (−)—Z-NHCH₂C(O)OH.

26. A method according to claim 25,
wherein said compound of formula I is (+)—Z-NHCH₂C(O)OH.

27. A method according to claim 23,
wherein said compound of formula I is Q-CH₂CH(CH₂(C₆H₅))C(O)OH.

28. A method according to claim 27,
wherein said compound of formula I is (3R,4R,S)-Q-CH₂CH(CH₂(C₆H₅))C(O)OH.

29. A method according to claim 1,
wherein said compound is a substantially pure stereoisomer.

30. A method according to claim 1,
wherein said compound of formula I is [[2(S)-[[4(R)-(3-hydroxyphenyl)-3(R),4-dimethyl-piperidinyl]methyl]-1-oxo-3-phenylpropyl]amino]acetic acid dihydrate.

31. A method according to claim 1,
wherein said composition further comprises at least one opioid.

32. A method according to claim 31,
wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol, and mixtures thereof.

33. A product produced by the method of claim 1.

34. A product produced by the method of claim 31.

35. A composition, comprising:
a. at least one compound of formula I or a pharmaceutically acceptable salt or stable polymorph thereof: 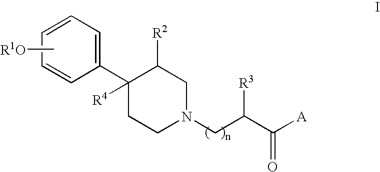
wherein:
R¹ is hydrogen or alkyl;
R²is hydrogen, alkyl, or alkenyl;
R³ is hydrogen, alkyl, alkenyl, aryl, cycloalkyl, cycloalkenyl, cycloalkyl-substituted alkyl, cycloalkenyl-substituted alkyl, or aralkyl;
R⁴ is hydrogen, alkyl, or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, aralkyl, or alkylene substituted B or, together with the nitrogen atom to which they are attached, R⁶ and R⁷ form a heterocyclic ring;
B is 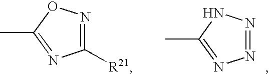
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 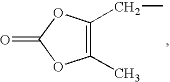
alkylene substituted (C═O)D, or —R¹³OC(═O)R¹⁴;
R¹³ is alkyl-substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl, or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl; and
n is 0 to 4;
b. a pharmaceutically-acceptable excipient selected from the group consisting of mannitol, dextrose, fructose, lactose, sucrose, dextrate, maltodextrin, and mixtures thereof; and
wherein said composition has an average particle size range of about 5 microns to about 20 microns.

36. A composition according to claim 35,
wherein said compound of formula I or a pharmaceutically acceptable salt or stable polymorph thereof has an average particle size range of about 5 microns to about 20 microns.

37. A composition according to claim 35,
wherein said pharmaceutically-acceptable excipient has an average particle size range of about 5 microns to about 20 microns.

38. A composition according to claim 35,
wherein the average particle size of said compound of formula I or a pharmaceutically acceptable salt or stable polymorph thereof differs from the average particle size of said excipient by no more than about 200%.

39. A composition according to claim 35,
wherein the average particle size of said compound of formula I or a pharmaceutically acceptable salt or stable polymorph thereof differs from the average particle size of said excipient by no more than about 100%.

40. A composition according to claim 35,
wherein the average particle size of said compound of formula I or a pharmaceutically acceptable salt or stable polymorph thereof differs from the average particle size of said excipient by no more than about 50%.

41. A composition according to claim 35,
wherein the average particle size of said compound of formula I or a pharmaceutically acceptable salt or stable polymorph thereof differs from the average particle size of said excipient by no more than about 25%.

42. A composition according to claim 35,
wherein the weight ratio of said compound of formula I or a pharmaceutically acceptable salt or stable polymorph thereof to said pharmaceutically acceptable excipient is about 10:1 to about 1:10.

43. A composition according to claim 35,
wherein the weight ratio of said compound of formula I or a pharmaceutically acceptable salt or stable polymorph thereof to said pharmaceutically acceptable excipient is about 5:1 to about 1:5.

44. A composition according to claim 35,
wherein the weight ratio of said compound of formula I or a pharmaceutically acceptable salt or stable polymorph thereof to said pharmaceutically acceptable excipient is about 2:1 to about 1:2.

45. A composition according to claim 35,
wherein the weight ratio of said compound of formula I or a pharmaceutically acceptable salt or stable polymorph thereof to said pharmaceutically acceptable excipient is about 1:1.

46. A composition according to claim 35, further comprising at least one opioid.

47. A composition according to claim 46,
wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, and tramadol.

48. A composition according to claim 46,
wherein said opioid has an average particle size range of about 5 microns to about 20 microns.

49. A composition according to claim 35,
wherein the compound of formula I is a trans 3,4-isomer.

50. A composition according to claim 35,
wherein:
R¹ is hydrogen;
R² is alkyl;
n is 1 or 2;
R³ is benzyl, phenyl, cyclohexyl, or cyclohexylmethyl; and
R⁴ is alkyl.

51. A composition according to claim 35,
wherein:
A is OR⁵; and
R⁵ is hydrogen or alkyl.

52. A composition according to claim 35,
wherein:
A is NR⁶R⁷;
R⁶ is hydrogen;
R⁷ is alkylene substituted B; and
B is C(O)W.

53. A composition according to claim 35,
wherein:
R⁷ is (CH₂)_{q}—B;
q is about 1 to about 3;
W is OR¹⁰; and
R¹⁰ is hydrogen, alkyl, phenyl-substituted alkyl, cycloalkyl, or cycloalkyl-substituted alkyl.

54. A composition according to claim 35,
wherein:
W is NR¹¹R¹²
R¹¹ is hydrogen or alkyl; and
R¹² is hydrogen, alkyl or alkylene substituted C(═O)Y.

55. A composition according to claim 35,
wherein:
R¹² is (CH₂)ₘC(O)Y;
m is 1 to 3;
Y is OR¹⁸ or NR¹⁹R²⁰; and
R¹⁸, R¹⁹, and R²⁰ are independently hydrogen or alkyl.

56. A composition according to claim 35,
wherein:
W is OE;
E is CH₂C(═O)D;
D is OR¹⁵ or NR¹⁶R¹⁷;
R¹⁵ is hydrogen or alkyl;
R¹⁶ is methyl or benzyl; and
R¹⁷ is hydrogen.

57. A composition according to claim 35,
wherein:
W is OE;
E is R¹³OC(═O)R¹⁴;
R¹³ is —CH(CH₃)— or —CH(CH₂CH₃)—; and
R¹⁴ is alkyl.

58. A composition according to claim 35,
wherein the configuration at positions 3 and 4 of the piperidine ring is each R.

59. A composition according to claim 35,
wherein said compound of formula I is selected from the group consisting of:
Q-CH₂CH(CH₂(C₆H₅))C(O)OH,
Q-CH₂CH₂CH(C₆H₅)C(O)NHCH₂C(O)OCH₂CH₂,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NHCH₂C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OCH₂CH₃,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NHCH₂C(O)OCH₂CH(CH₃)₂,
Z-NH(CH₂)₂C(O)OCH₂(C₆H₅),
Z-NH(CH₂)₂C(O)OH,
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)O(CH₂)₄CH₃,
Z-NHCH₂C(O)OCH₂C(O)NHCH₃,
Z-NHCH2C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
wherein:
Q represents 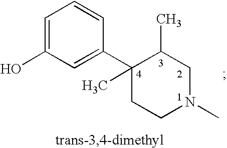
G represents 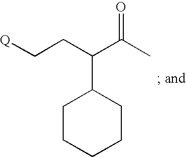
Z represents

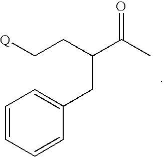

60. A composition according to claim 59,
wherein said compound of formula I is selected from the group consisting of:
(3R,4R,S)—Z-NHCH₂C(O)OCH₂CH(CH₃)₂,
(+)—Z-NHCH₂C(O)OH,
(−)—Z-NHCH₂C(O)OH,
(3R,4R,R)—Z-NHCH₂C(O)—OCH₂CH(CH₃)₂,
(3S,4S,S)—Z-NHCH₂C(O)OCH₂CH(CH₃)₂,
(3S,4S,R)—Z-NHCH₂C(O)OCH₂CH(CH₃)₂,
(3R,4R)—Z-NHCH₂C(O)NHCH₂(C₆H₅) and
(3R,4R)-G-NH(CH₂)₃C(O)OH.

61. A composition according to claim 60,
wherein said compound of formula I is selected from the group consisting of (+)—Z-NHCH₂C(O)OH and (−)—Z-NHCH₂C(O)OH.

62. A composition according to claim 61,
wherein said compound of formula I is (+)—Z-NHCH₂C(O)OH.

63. A composition according to claim 60,
wherein said compound of formula I is Q-CH₂CH(CH₂(C₆H₅))C(O)OH.

64. A composition according to claim 60,
wherein said compound of formula I is (3R,4R,S)-Q-CH₂CH(CH₂(C₆H₅))C(O)OH.

65. A composition according to claim 35,
wherein said compound of formula I is a substantially pure stereoisomer.

66. A solid dosage form, comprising:
a composition according to claim 35.

67. A solid dosage form according to claim 66,
wherein said solid dosage form is a capsule.

68. A solid dosage form according to claim 66,
wherein said solid dosage form is a tablet.

69. A method of preventing or treating a side effect associated with an opioid in a patient, comprising the step of:
administering to said patient an effective amount of the composition of claim 35.

70. A method according to claim 69,
wherein said side effect is ileus, pruritis, constipation, urinary retention, biliary spasm, opioid bowel dysfunction, colic, nausea, or vomiting or a combination thereof.

71. A method according to claim 70,
wherein said side effect is postoperative ileus, postpartum ileus, pruritis, constipation, urinary retention, biliary spasm, opioid bowel dysfunction, colic, postoperative nausea, or postoperative vomiting of a combination thereof.

72. A method of treating or preventing pain in a patient, comprising the step of:
administering to said patient in need thereof an effective amount of the composition of claim 35.

73. A method according to claim 72,
wherein said composition further comprises at least one opioid.

74. A method according to claim 73,
wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol, and mixtures thereof.
